SEQUENCE LISTING (32 sequences)

SEQ ID NO: 1 - protein, 8 aa, Artificial Sequence
  Description of Artificial Sequence: Synthetic peptide
  MOD_RES (2)..(2): Trp or Ala
  MOD_RES (3)..(3): Leu or Thr
  MOD_RES (4)..(4): Leu, Ala, Asp or Phe
  MOD_RES (6)..(6): Leu, Gly or Thr
  MOD_RES (7)..(7): Phe, Leu or Met
YXXXLXXV

SEQ ID NO: 2 - protein, 10 aa, Artificial Sequence
  Description of Artificial Sequence: Synthetic peptide
  MOD_RES (3)..(3): Leu or Asp
  MISC_FEATURE (6)..(7): This region may encompass “Gln Trp” or “Arg Phe”
KDXMVXXFKD

SEQ ID NO: 3 - protein, 13 aa, Artificial Sequence
  Description of Artificial Sequence: Synthetic peptide
  MOD_RES (7)..(7): Trp or Ala
  MOD_RES (8)..(8): Leu or Thr
  MOD_RES (9)..(9): Leu, Ala, Asp or Phe
  MOD_RES (11)..(11): Leu, Gly or Thr
  MOD_RES (12)..(12): Phe, Leu or Met
MANLGYXXXLXXV

SEQ ID NO: 4 - protein, 28 aa, Artificial Sequence
  Description of Artificial Sequence: Synthetic CPP-IRF5 peptide
MANLGYWLLLLFVTMWTDVGLAKKRPKP

SEQ ID NO: 5 - protein, 28 aa, Artificial Sequence
  Description of Artificial Sequence: Synthetic CPP-IRF5 peptide
MANLGYWLALLFVTMWTDVGLFKKRPKP

SEQ ID NO: 6 - protein, 28 aa, Artificial Sequence
  Description of Artificial Sequence: Synthetic CPP-IRF5 peptide
MANLGYWLLALFVTYWTDLGLVKKRPKP

SEQ ID NO: 7 - protein, 28 aa, Artificial Sequence
  Description of Artificial Sequence: Synthetic CPP-IRF5 peptide
MANLGYWLYALFLTMVTDVGLFKKRPKP

SEQ ID NO: 8 - protein, 21 aa, Artificial Sequence
  Description of Artificial Sequence: Synthetic CPP-IRF5 peptide
KDLMVQWFKDGGPSSGAPPPS

SEQ ID NO: 9 - protein, 31 aa, Artificial Sequence
  Description of Artificial Sequence: Synthetic CPP-IRF5 polypeptide
IRLQISNPDLKDLMVQWFKDGGPSSGAPPPS

SEQ ID NO: 10 - protein, 30 aa, Artificial Sequence
  Description of Artificial Sequence: Synthetic CPP-IRF5 polypeptide
PFPPLPIGEEAPKDDMVRFFKDLHQYLNVV

SEQ ID NO: 11 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 12 - skipped

SEQ ID NO: 13 - protein, 23 aa, Artificial Sequence
  Description of Artificial Sequence: Synthetic CPP-IRF5 peptide
IRLQISNPYLKFIPLKRAIWLIK

SEQ ID NO: 14 - protein, 20 aa, Artificial Sequence
  Description of Artificial Sequence: Synthetic CPP-IRF5 peptide
MIILIISFPKHKDWKVILVK

SEQ ID NO: 15 - protein, 19 aa, Artificial Sequence
  Description of Artificial Sequence: Synthetic peptide
  MOD_RES (2)..(2): Any amino acid
  MOD_RES (4)..(4): Any amino acid
  MOD_RES (7)..(7): Any amino acid
  MOD_RES (9)..(10): Any amino acid
  MOD_RES (13)..(14): Any amino acid
  MOD_RES (16)..(18): Any amino acid
IXLXISXPXXKDXXVXXXK

SEQ ID NO: 16 - protein, 24 aa, Artificial Sequence
  Description of Artificial Sequence: Synthetic FITC tagged CPP-IRF5 peptide
  MOD_RES (1)..(1): Aha
XIRLQISNPYLKFIPLKRAIWLIK

SEQ ID NO: 17 - protein, 21 aa, Artificial Sequence
  Description of Artificial Sequence: Synthetic FITC tagged CPP-IRF5 peptide
  MOD_RES (1)..(1): Aha
XMIILIISFPKHKDWKVILVK

SEQ ID NO: 18 - protein, 29 aa, Artificial Sequence
  Description of Artificial Sequence: Synthetic FITC tagged CPP-IRF5 peptide
  MOD_RES (1)..(1): Aha
XMANLGYWLLLLFVTMWTDVGLAKKRPKP

SEQ ID NO: 19 - protein, 29 aa, Artificial Sequence
  Description of Artificial Sequence: Synthetic FITC tagged CPP-IRF5 peptide
  MOD_RES (1)..(1): Aha
XMANLGYWLALLFVTMWTDVGLFKKRPKP

SEQ ID NO: 20 - protein, 29 aa, Artificial Sequence
  Description of Artificial Sequence: Synthetic FITC tagged CPP-IRF5 peptide
  MOD_RES (1)..(1): Aha
XMANLGYWLLALFVTYWTDLGLVKKRPKP

SEQ ID NO: 21 - protein, 29 aa, Artificial Sequence
  Description of Artificial Sequence: Synthetic FITC tagged CPP-IRF5 peptide
  MOD_RES (1)..(1): Aha
XMANLGYWLYALFLTMVTDVGLFKKRPKP

SEQ ID NO: 22 - protein, 210 aa, Artificial Sequence
  Description of Artificial Sequence: Synthetic N-terminal His (six histadine)-tagged IRF5 (222-425) polypeptide
HHHHHHPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARL

SEQ ID NO: 23 - protein, 15 aa, Artificial Sequence
  Description of Artificial Sequence: Synthetic CPP-IRF5 peptide
YLKFIPLKRAIWLIK

SEQ ID NO: 24 - protein, 8 aa, Artificial Sequence
  Description of Artificial Sequence: Synthetic FLAG-tag peptide
DYKDDDDK

SEQ ID NO: 25 - protein, 9 aa, Artificial Sequence
  Description of Artificial Sequence: Synthetic HA-tag peptide
YPYDVPDYA

SEQ ID NO: 26 - protein, 10 aa, Artificial Sequence
  Description of Artificial Sequence: Synthetic Myc-tag peptide
EQKLISEEDL

SEQ ID NO: 27 - protein, 38 aa, Artificial Sequence
  Description of Artificial Sequence: Synthetic SBP-tag polypeptide
MDEKTTGWRGGHVVEGLAGELEQLRARLEHHPQGQREP

SEQ ID NO: 28 - protein, 14 aa, Artificial Sequence
  Description of Artificial Sequence: Synthetic V5-tag peptide
GKPIPNPLLGLDST

SEQ ID NO: 29 - protein, 8 aa, Artificial Sequence
  Description of Artificial Sequence: Synthetic peptide
  MOD_RES (2)..(2): Trp or Ala
  MOD_RES (3)..(3): Leu or Thr
  MOD_RES (4)..(4): Leu, Ala, Asp, Phe or Tyr
  MOD_RES (5)..(5): Leu or Ala
  MOD_RES (6)..(6): Leu, Gly or Thr
  MOD_RES (7)..(7): Phe, Leu or Met
  MOD_RES (8)..(8): Val or Leu
YXXXXXXX

SEQ ID NO: 30 - protein, 11 aa, Artificial Sequence
  Description of Artificial Sequence: Synthetic peptide
  MOD_RES (2)..(2): Any amino acid
  MOD_RES (4)..(4): Any amino acid
  MOD_RES (7)..(7): Any amino acid
  MOD_RES (9)..(9): Any amino acid
  MOD_RES (10)..(10): Any amino acid
IXLXISXPXXK

SEQ ID NO: 31 - protein, 8 aa, Artificial Sequence
  Description of Artificial Sequence: Synthetic peptide
  MOD_RES (2)..(2): Trp or Ala
  MOD_RES (3)..(3): Leu or Thr
  MOD_RES (4)..(4): Leu, Ala, Asp, Phe or Tyr
  MOD_RES (6)..(6): Leu, Gly or Thr
  MOD_RES (7)..(7): Phe, Leu or Met
  MOD_RES (8)..(8): Val or Leu
YXXXLXXX

SEQ ID NO: 32 - protein, 6 aa, Artificial Sequence
  Description of Artificial Sequence: Synthetic 6xHis tag
HHHHHH

(19) United States
(12) Patent Application Publication
(10) Pub. No.: US 2016/0009772 A1
(43) Pub. Date: Jan. 14, 2016

(54) CELL PENETRATING PEPTIDES WHICH BIND IRF5

(71) Applicant: HOFFMANN-LA ROCHE INC., Nutley, NJ (US)

(72) Inventors: Julie Demartino, Westfield, NJ (US); Nader Fotouhi, Basking Ridge, NJ (US); Ann Hoffman, Mahwah, NJ (US); Kuo-Sen Huang, Livingston, NJ (US); Francesca Milletti, New York, NY (US); Sandip Panicker, Morristown, NJ (US); Dinesh Srinivasan, Freemont, CA (US); Seng-Lai Tan, Sudbury, MA (US)

(21) Appl. No.: 14/432,977

(22) PCT Filed: Oct. 7, 2013
(86) PCT No.: PCT/EP2013/070759

§ 371 Date: Apr. 1, 2015

Related U.S. Application Data
(60) Provisional application No. 61/710,817, filed on Oct. 8, 2012.

(51) Int. Cl. C07K 14/47; G01N 21/64; G01N 33/68

(52) U.S. Cl. CPC C07K 14/47; G01N 33/6872; G01N 21/6408; G01N 21/6428; G01N 2500/20; G01N 2500/04

(57) ABSTRACT

The present invention comprises cell penetrating peptides that bind to interferon regulatory factor IRF5 and disrupt the IRF5 homo-dimerization and/or attenuate downstream signaling, and a method for screening peptides that inhibit IRF5. Generally, the cell penetrating peptides of the invention bind human interferon regulatory factor IRF5 (CPP-IRF5).

### FIELD OF THE INVENTION

The present invention comprises cell penetrating peptides that bind to interferon regulatory factor 5 (IRF5) and disrupt the IRF5 homo-dimerization and/or attenuate downstream signaling, and a method for screening for said peptides that inhibit IRF5.

### BACKGROUND OF THE INVENTION

IRF5 is a putative therapeutic target that regulates key components of autoimmune etiology, including systemic lupus erythematosus (SLE) and downstream regulation of IL6 and IL12. Multiple genome wide association studies (GWAS) report that IRF5 polymorphisms are associated with an increased risk of SLE and existing pre-clinical literature together provide compelling rationale that blocking IRF5 function may be beneficial to SLE patients (Agarwal; Cunninghame et al.; Demirci et al.; Dieudé and Dawidowicz). Data presented in pre-clinical literature provide important clues to the critical role of IRF5 in regulating key components of autoimmune disease etiology. However, the absence of specific tools targeting IRF5 have limited early target evaluation efforts to use of siRNA against IRF5 or using knockout IRF5 mice (Beal; Feng et al.; Kozyrev and Alarcon; Krausgruber et al.; Lien et al.).

In addition, blocking IRF5 function would impact Toll like receptor 7/8/9 signaling in cell types relevant to SLE that express IRF5 (Monocytes, macrophages, plasmacytoid dendritic cells and B cells). Thus targeting IRF5 may significantly benefit patients with SLE or other autoimmune diseases wherein IRF5 signaling plays a significant role by attenuating dysregulated signaling for eg. TLR7/8/9 signaling resulting in interferon production by pDC, IL-12, IL6 and TNFα by monocytes/macrophages as well as autoantibody production by B cells.

Cell-penetrating peptides (CPPs) are a class of peptides with the ability to convey various, otherwise impermeable, macromolecules across the plasma membrane of cells in a relatively non-toxic fashion. The CPP peptides are typically between 5 and about 30 amino acids (aa) in length with a cationic, amphipathic, or hydrophobic nature. Notable examples of cell-penetrating peptides include Tat, Penetratin, and Transportan. (Fawell, S. et al. Proc. Natl. Acad. Sci. 1994, pp 664-668; Theodore, L. et al. J. Neurosci. 1995, pp 7158-7167; Pooga, M. et al. FASEB J. 1998, pp 67-77). A cell penetrating peptide such as Tat can be attached to an effector peptide, or the effector peptide can be intrinsically cell-penetrating. Examples of effector peptides intrinsically cell-penetrating include Arf(1-22) and p28, among others (Johansson, H. J. et al. Mol. Ther. 2007, 16(1), pp 115-123; Taylor, B. N. et al. Cancer Res. 2009, 69 (2), pp. 537-546).

In order to properly dissect the role of IRF5 and to inhibit the dimerization of the target, so-called tool molecules (small molecules or peptides) are necessary. Though the crystal structure of IRF5 is known (Chen et al.), there has been a lack of specific tools (small molecules or peptides) which both target IRF5 and inhibit homo-dimerization, thus regulating the function of IRF5.

The present invention focuses on novel cell-penetrating peptides designed to both reach the target and inhibit residues critical for dimer formation (a key step regulating nuclear translocation and function). Due to the lack of a direct approach to biochemically evaluate such cell-penetrating peptides and other tool molecules targeting IRF5 dimerization, a novel FRET based biochemical assay was established. The biochemical assay described in this patent identifies tools that inhibit dimerization of IRF5.

The present invention thus generally relates to peptides that are cell-penetrating and with the ability to bind to IRF5 and disrupt the IRF5 homo-dimerization and/or attenuate downstream signaling as well as methods of testing, screening and evaluating peptides, specifically cell-penetrating peptides, which bind to and/or inhibit IRF5.

### BRIEF DESCRIPTION OF THE FIGURES

FIG. 1: The figure depicts the principle and validation of an exemplary FRET dimer assay of the invention. FIG. 1A provides a schematic of the biochemical IRF5 FRET dimer assay described in detail in Example 12. The graphs 1B & 1C serve to validate the use of the biochemical assay. In these experiments, biotin tag IRF5 proteins (200 nM) were mixed with equal volume of increasing amounts of 6-His (hereinafter His) tag IRF5 proteins. The abscissa represents concentrations of the his-tagged IRF5 and the ordinate represents the measured value of the TR-FRET that occurs. It has been suggested in the literature (Royer et al., 2010) that phosphorylation of 5430 facilitates dimerization of IRF5. The S430D mutant is considered to be phosphomimetic. In FIG. 1B, the Kd (μM) indicate that the rank order of dimerization is S430D:S430D<S430D:Wild Type<Wild type:Wild Type and these observations are in a agreement with values reported in literature. FIG. 1C shows that the TR_FRET signals decrease significantly with the S430D+R353D mutants and are also in alignment with expected behavior of IRF5 dimerization.

FIG. 2: This figure depicts the labeled peptides of the invention binding IRF5. FIG. 2A provides a schematic of the biochemical direct binding FRET assay (FITC CPP Binding to IRF5 (222-425)) described in detail in Example 13. In these experiments, labeled cell-penetrating peptides of the invention, specifically FITC-labeled versions of SEQ ID NOS 13-14 and 4-7 (100 nM) (SEQ ID NOS 16-21) were mixed with increasing concentrations of the N-terminal His (six histadine)-tagged IRF5 (222-425) SEQ ID NO 22. The abscissa in the graphs 2B & 2C below represents concentrations (μM) of the his-tagged IRF5 (222-425, lacking helix 5 and unable to homodimerize) and the ordinate represents the measured value of the TR-FRET in the presence of the FITC labeled versions of SEQ ID NOS 13-14 and 4-7 (SEQ ID NOS 16-21). The Kds indicate that all six FITC versions of SEQ ID NOS 13-14 and 4-7 (SEQ ID NOS 16-21) directly bind the IRF5 protein tested. A control FITC labeled CPP designed not to bind IRF5 (SEQ ID NO: 23) does not display any affinity.

FIG. 3: This figure shows localization of FITC-labeled CPP's (CPP are SEQ ID NOS: 13-14 and 4-7, FITC-labeled versions of SEQ ID NOS 13-14 and 4-7 are SEQ ID NOS 16-21) after 2 hours (h) (FIG. 3A) and 24 hours (h) (FIG. 3B) incubation in HeLa cells, and thus confirms that the CPPs tested are cell penetrant. The protocol used is described in detail in Example 14. Briefly, HeLa cells were treated for 2 h and 24 h time points (panel A and panel B respectively) with 10 μM or 3 μM concentrations of the FITC tagged CPPs. The images were obtained at 40× magnification.

FIG. 4: This figure shows attenuation of IL-6 production in THP-1 (human monocytic cell line) by SEQ ID NOS 13-14 and 4-7 compared against a control (V). THP-1 cells were pre-treated with 50 μM of these CPP's (SEQ ID NOS: 13-14 and 4-7) for 30 min and stimulated o/n with 10 μM of R848 (TLR7/8 agonist) as described in Example 15. The abscissa represents the treatment conditions and the ordinate represents the amount of IL6 produced normalized to the vehicle (no peptide but stimulated with 10 μM R848) and cell number as measured by cell titer glo. SEQ ID NOS: 13-14 and 4-7 all attenuate the R848 stimulated IL6 production.

FIGS. 5A-5F: These figures depict attenuation of R848 induced IL-12 production in human peripheral blood mononuclear cells (PMBC) by SEQ ID NOS 13-14 and 4-7 in a concentration dependent manner. PBMC's isolated from healthy human volunteers were pre-treated with SEQ ID NOS 13-14 and 4-7 for 30 min and stimulated o/n with 1 μM of R848 (TLR7/8 agonist) as described in Example 16. The abscissa represents the concentrations of compound used and the ordinate represents the amount of IL12. SEQ ID NOS 13-14 and 4-7 all attenuate the R848 stimulated IL12 production from PBMC's in a concentration dependent manner. The rank order of potency is SEQ ID NO 14<SEQ ID NO 4<SEQ ID NO 6<SEQ ID NO 5<SEQ ID NO 7<SEQ ID NO 13.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

The nomenclature used in this Application is based on IUPAC systematic nomenclature, unless indicated otherwise.

All peptide sequences mentioned herein are written according to the usual convention whereby the N-terminal amino acid is on the left and the C-terminal amino acid is on the right, unless noted otherwise. A short line between two amino acid residues indicates a peptide bond. Where the amino acid has isomeric forms, it is the L form of the amino acid that is represented unless otherwise expressly indicated.

The term “Amino acid” denotes an organic compound of general formula NH₂CHRCOOH where R can be any organic group. Specifically, the term amino acid may refer to natural and unnatural (man-made) amino acids. For convenience in describing this invention, the conventional and nonconventional abbreviations for the various amino acids residues are used. These abbreviations are familiar to those skilled in the art, but for clarity are listed below: Asp=D=Aspartic Acid; Ala=A=Alanine; Arg=R=Arginine; Asn=N=Asparagine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Nle=Norleucine; Phe=F=Phenylalanine; Pro=P=Proline; Ser=S=Serine; Thr=T=Threonine; Trp=W=Tryptophan; Tyr=Y=Tyrosine; and Val=V=Valine.

The term “Amino acid motif” denotes a conserved sequence of amino acids (e.g. Y---L--V). This sequence may also include gaps to indicate the number of residues that separate each amino acid of the motif.

A cell-penetrating peptide (CPP) of the invention denotes a peptide of about 5 to about 30 amino acids, without a conformational restriction in the form of a bridge or cyclic peptide created by joining two or more unnatural amino acides (i.e., it is not a “stapled peptide”), and which is able to penetrate cell membranes (for example to translocate different cargoes into cells).

The phrase “peptide(s) which bind IRF5” or “peptide(s) which is/are capable of binding IRF5” denotes those groups of peptides which are positive (defined herein as where IC50<75 uM) in a biochemical assay where the target is IRF5.

The term “IRF5” (interferon regulatory factor 5) denotes a protein, comprising generally of amino acid sequence

(Isoform 1, SEQ ID NO: 11)
MNQSIPVAPTPPRRVRLKPWLVAQVNSCQYPGLQWVNGEKKLFCIPWRHA
TRHGPSQDGDNTIFKAWAKETGKYTEGVDEADPAKWKANLRCALNKSRDF
RLIYDGPRDMPPQPYKIYEVCSNGPAPTDSQPPEDYSFGAGEEEEEEEEL
QRMLPSLSLTEDVKWPPTLQPPTLRPPTLQPPTLQPPVVLGPPAPDPSPL
APPPGNPAGFRELLSEVLEPGPLPASLPPAGEQLLPDLLISPHMLPLTDL
EIKFQYRGRPPRALTISNPHGCRLFYSQLEATQEQVELFGPISLEQVRFP
SPEDIPSDKQRFYTNQLLDVLDRGLILQLQGQDLYAIRLCQCKVFWSGPC
ASAHDSCPNPIQREVKTKLFSLEHFLNELILFQKGQTNTPPPFEIFFCFG
EEWPDRKPREKKLITVQVVPVAARLLLEMFSGELSWSADSIRLQISNPDL
KDRMVEQFKELHHIWQSQQRLQPVAQAPPGAGLGVGQGPWPMHPAGMQ.
Alternatively, IRF5 denotes other iso forms,
including at least Isoform 2, 3, or 4.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The terms “pharmaceutical composition” and “pharmaceutical formulation” (or “formulation”) are used interchangeably and denote a preparation which is in such form as to permit the biological activity of an active ingredient contained therein to be effective, and which contains no additional components which are unacceptably toxic to a subject to which the pharmaceutical composition would be administered.

A “liquid composition” denotes a composition which is aqueous or liquid at a temperature of at least about 2 to about 8° C. under atmospheric pressure.

The term “lyophilization” denotes the process of freezing a substance and then reducing the concentration of water, by sublimation and/or evaporation to levels which do not support biological or chemical reactions.

The term “lyophilized composition” (or “lyocomposition”) denotes a composition that is obtained or obtainable by the process of lyophilization of a liquid composition. Typically it is a solid composition having a water content of less than 5%.

The term “reconstituted composition” denotes a lyophilized composition which is combined with reconstitution medium that promotes dissolution of the lyophilized composition. Examples of reconstitution medium include, but are not limited to, water for injection (WFI), bacteriostatic water for injection (BWFI), sodium chloride solutions (e.g. 0.9% (w/v) NaCl), glucose solutions (e.g. 5% glucose), surfactant comprising solutions (e.g. 0.01% polysorbate 20), or pH-buffered solution (e.g. phosphate-buffered solutions).

The term “sterile” denotes that a composition or excipient has a probability of being microbially contaminated of less than 10e-6.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “half maximal inhibitory concentration” (IC50) denotes the concentration of a particular compound or molecule required for obtaining 50% inhibition of a biological process in vitro. IC50 values can be converted logarithmically to pIC50 values (−log IC50), in which higher values indicate exponentially greater potency. The IC50 value is not an absolute value but depends on experimental conditions e.g. concentrations employed. The IC50 value can be converted to an absolute inhibition constant (Ki) using the Cheng-Prusoff equation (Biochem. Pharmacol. (1973) 22:3099).

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The terms “N-terminal modification” and “amino group modification” are used interchangeably to denote the addition of a functional group at the N terminus of a peptide or protein. Particularly, N-terminal modifications are posttranslational. Examples for N-terminal modifications are commonly known in the art such as acetylation, pyroglutamate formation, myristoylation, methylation, carbamylation, or formylation. Particular N-terminal modification is acetylation.

The terms “C-terminal modification” and “carboxyl group modification” are used interchangeably to denote the addition of a functional group at the C terminus of a peptide or protein. Particularly, C-terminal modifications are posttranslational. Examples for C-terminal modifications are commonly known in the art such as amidation, prenylation, glypiation, ubiquitination, sumoylation, or methyl/ethyl-esterification. Particular C-terminal modification is amidation.

For convenience, and readily known to one skilled in the art, the following abbreviations or symbols are used to represent the moieties, reagents and the like used and/or referenced in this invention:
μl microliters
μM micromolar
Ac acetyl
Aha Amino hexanoic acid
Alexa a family of fluorescence dyes produced by Invitrogen Corp
APC Allophycocyanin
BOP benzotriazol-1-yloxy-tris-(dimethylamino)phosphonium-hexafluorophosphate
BSA Bovine serum albumin
CPP Cell Penetrating Peptide
DIPEA N,N-diisopropylethylamine
DMF dimethylformamide
DMSO Dimethyl Sulfoxide
DTT Dithiothreitol
DyLight A family of fluorescence dyes produced by Dyomics
ES-MS electro spray mass spectrometry
Et₂O diethyl ether
Eu Europium
Eu Europium
FAB-MS fast atom bombardment mass spectrometry
FITC Fluorscein isothiocyanate
FLAG-tag a peptide recognized by an antibody (DYKDDDDK) (SEQ ID NO:24)
Fmoc 9-fluorenylmethyloxycarbonyl
FRET Forster/Fluorescence resonance energy transfer
GST Glutathione S-transferase
GWAS Genome Wide Association Studies
h Hour
HA-tag a peptide recognized by an antibody (YPYDVPDYA) (SEQ ID NO:25)
HBTU 2-(1H-benzotriazole-1-yl)-1,1,3,3-tetramethyluronium-hexafluorophosphate
His-tag Hexa histidine tag
HOBT N-hydroxybenzotriazole
hr(s) hour(s)
IC50 Half maximal inhibitory concentration
IL-12 Interleukin 12
IL-12p40 Interleukin 12 subunit p40
IL6 Interleukin 6
IRB Institutional review board
IRF5 Interferon regulatory factor 5
min Minutes
Myc-tag a short peptide recognized by an antibody (EQKLISEEDL) (SEQ ID NO:26)
NaCl Sodium Chloride
NFkB Nuclear factor kappa-light-chain-enhancer of activated B cells
nm Nanometer
NMP N-methyl-pyrrolidone
PBMC Peripheral blood mononuclear cells
PBS Phosphate buffered saline
R848 Resiquimod, 1-[4-amino-2-(ethoxymethyl)imidazo[4,5-c]quinolin-1-yl]-2-methylpropan-2-ol
Ru Ruthenium
SBP-tag a peptide which binds to streptavidin (MDEKTTGWRGGHVVEGLAGELEQLRARLEHHPQGQREP) (SEQ ID NO:27)
siRNA Small interfering RNA
SLE Systemic Lupus Erythematosus
SSA succinimidyl succinamide
TAMRA carboxytetramethylrhodamine
Tb Terbium
TFA trifluoroacetic acid
TIS triisopropylsilane
TLR Toll-like receptor
TR-FRET Time-resolved FRET
Tris-HC tris(hydroxymethyl)amino methane
V5-tag a peptide recognized by an antibody (GKPIPNPLLGLDST) (SEQ ID NO:28)
WT Wildtype

### DETAILED DESCRIPTION OF THE INVENTION

The present invention provides compounds which are cell-penetrating peptides that inhibit interferon regulatory factor IRF5 by targeting IRF5 (homo)dimerization.

In a general embodiment, the compounds are cell-penetrating peptides which bind interferon regulatory factor IRF5 (CPP-IRF5), wherein the peptides comprise an amino acid sequence of 20 to 40 amino acids and wherein said amino acid sequence further comprises an amino acid sequence motif selected from the group consisting of

a) I-x-L-x-I-S-x-P-x-x-K (SEQ ID NO: 25), wherein
I is isoleucine,
L is leucine,
S is serine,
P is proline,
K is lysine, and
x is independently selected from any amino acid; or

b) Y-R1-R2-R3-R8-R4-R5-R9 (SEQ ID NO: 24), wherein
Y is tyrosine,
R1 is an amino acid selected from the group of tryptophan (W) or alanine (A),
R2 is an amino acid selected from the group consisting of leucine (L) or threonine (T),
R3 is an amino acid selected from the group consisting of leucine (L), alanine (A), aspartic acid (D), phenylalanine (F), or tyrosine (Y),
R8 is leucine (L) or alanine (A),
R4 is an amino acid selected from the group consisting of leucine (L), glycine (G) or threonine (T),
R5 is an amino acid selected from the group consisting of phenylalanine (F), leucine (L) or methionine (M), and
R9 is valine (V) or leucine (L); or

c) K-D-R6-M-V-R7-F-K-D (SEQ ID NO: 2), wherein
K is lysine,
D is aspartic acid,
R6 is an amino acid selected from the group consisting of leucine or aspartic acid,
M is methionine,
R7 is selected from the group consisting of Glutamine-Tryptophan (Q-W) and arginine-phenylalanine (R-F), and
F is phenylalanine;

or pharmaceutically acceptable salts thereof.

In one embodiment, the compounds are CPP-IRF5 peptides as described above, wherein the peptides comprise an amino acid sequence of 20 to 40 amino acids and wherein said amino acid sequence further comprises an amino acid sequence motif selected from the group consisting of

a) Y-R1-R2-R3-L-R4-R5-V (SEQ ID NO: 1), wherein
Y is tyrosine,
R1 is an amino acid selected from the group of tryptophan (W) or alanine (A),
R2 is an amino acid selected from the group consisting of leucine (L) or threonine (T),
R3 is an amino acid selected from the group consisting of leucine (L), alanine (A), aspartic acid (D), or phenylalanine (F),
L is leucine,
R4 is an amino acid selected from the group consisting of leucine (L), glycine (G) or threonine (T),
R5 is an amino acid selected from the group consisting of phenylalanine (F), leucine (L) or methionine (M), and
V is valine; or

b) K-D-R6-M-V-R7-F-K-D (SEQ ID NO: 2), wherein
K is lysine,
D is aspartic acid,
R6 is an amino acid selected from the group consisting of leucine or aspartic acid,
M is methionine,
R7 is selected from the group consisting of Glutamine-Tryptophan (Q-W) and arginine-phenylalanine (R-F), and
F is phenylalanine;

or pharmaceutically acceptable salts thereof.

A particular embodiment of the present invention relates to CPP-IRF5 peptides as described above which comprise an amino acid sequence of 20 to 35 amino acids.

In one embodiment, the compounds are cell-penetrating peptides which bind interferon regulatory factor IRF5 (CPP-IRF5), wherein the peptides comprise an amino acid sequence of 20 to 40 amino acids, particularly 20 to 35 amino acids, and wherein said amino acid sequence further comprises an amino acid sequence motif

I-x-L-x-I-S-x-P-x-x-K (SEQ ID NO: 25), wherein
I is isoleucine,
L is leucine,
S is serine,
P is proline,
K is lysine, and
x is any amino acid,

or pharmaceutically acceptable salts thereof.

Another particular embodiment of the present invention relates to CPP-IRF5 peptides as described above, wherein the amino acid sequence motif is I-x-L-x-I-S-x-P-x-x-K (SEQ ID NO: 25), wherein x is as defined above.

In a particular embodiment of the invention, x is independently selected from any natural amino acid. More particularly, x is independently selected from the group of arginine (R), asparagine (N), glutamine (Q), histidine (H), isoleucine (I), leucine (L), lysine (K), phenylalanine (F), and tyrosine (Y).

In one embodiment, the compounds are cell-penetrating peptides which bind interferon regulatory factor IRF5 (CPP-IRF5), wherein the peptides comprise an amino acid sequence of 20 to 40 amino acids, particularly 20 to 35 amino acids, and wherein said amino acid sequence further comprises an amino acid sequence motif

Y-R1-R2-R3-R8-R4-R5-R9 (SEQ ID NO: 24), wherein
Y is tyrosine,
R1 is an amino acid selected from the group of tryptophan (W) or alanine (A),
R2 is an amino acid selected from the group consisting of leucine (L) or threonine (T),
R3 is an amino acid selected from the group consisting of leucine (L), alanine (A), aspartic acid (D), phenylalanine (F), or tyrosine (Y),
R8 is leucine (L) or alanine (A),
R4 is an amino acid selected from the group consisting of leucine (L), glycine (G) or threonine (T),
R5 is an amino acid selected from the group consisting of phenylalanine (F), leucine (L) or methionine (M), and
R9 is valine (V) or leucine (L),

or pharmaceutically acceptable salts thereof.

Another particular embodiment of the present invention relates to CPP-IRF5 peptides as described above, wherein the amino acid sequence motif is Y-R1-R2-R3-R8-R4-R5-R9 (SEQ ID NO: 24), wherein R1, R2, R3, R4, R5, R8 and R9 are as defined above.

Another particular embodiment of the present invention relates to CPP-IRF5 peptides as described above, wherein the amino acid sequence motif is Y-R1-R2-R3-L-R4-R5-V (SEQ ID NO: 1), wherein R1, R2, R3, R4 and R5 are as defined above.

Another particular embodiment of the present invention relates to CPP-IRF5 peptides as described above, wherein the amino acid sequence motif is MANLG-Y-R1-R2-R3-L-R4-R5-V (SEQ ID NO: 3), wherein M is methionine, A is alanine, N is asparagine, L is leucine, G is glycine, Y is tyrosine, V is valine and R1, R2, R3, R4, and R5 are as defined above.

In one embodiment, the compounds are cell-penetrating peptides which bind interferon regulatory factor IRF5 (CPP-IRF5), wherein the peptides comprise an amino acid sequence of 20 to 40 amino acids, particularly 20 to 35 amino acids, and wherein said amino acid sequence further comprises an amino acid sequence motif

K-D-R6-M-V-R7-F-K-D (SEQ ID NO: 2), wherein
K is lysine,
D is aspartic acid,
R6 is an amino acid selected from the group consisting of leucine or aspartic acid,
M is methionine,
R7 is selected from the group consisting of Glutamine-Tryptophan (Q-W) and arginine-phenylalanine (R-F), and
F is phenylalanine,

or pharmaceutically acceptable salts thereof.

Another particular embodiment of the present invention relates to CPP-IRF5 peptides as described above, wherein the amino acid sequence motif is K-D-R6-M-V-R7-F-K-D (SEQ ID NO: 2), wherein R6 and R7 are as defined above.

Another particular embodiment of the present invention relates to CPP-IRF5 peptides as described above, additionally comprising a second peptide which is a cell penetrating peptide (CPP).

Another particular embodiment of the present invention relates to CPP-IRF5 peptides as described above, additionally comprising an N-terminal modification and/or a C-terminal modification.

Another more particular embodiment of the present invention relates to CPP-IRF5 peptides as described above, additionally comprising an N-terminal modification selected from acetylation and/or a C-terminal modification selected from amidation.

Another particular embodiment of the present invention relates to CPP-IRF5 peptides as described above, wherein the peptides comprise an amino acid sequence selected from the group consisting of

SEQ ID NO 13:
IRLQISNPYLKFIPLKRAIWLIK,
SEQ ID NO 14:
MIILIISFPKHKDWKVILVK,
SEQ ID NO 4:
MANLGYWLLLLFVTMWTDVGLAKKRPKP,
SEQ ID NO 5:
MANLGYWLALLFVTMWTDVGLFKKRPKP,
SEQ ID NO 6:
MANLGYWLLALFVTYWTDLGLVKKRPKP,
SEQ ID NO 7:
MANLGYWLYALFLTMVTDVGLFKKRPKP,
SEQ ID NO 8:
KDLMVQWFKDGGPSSGAPPPS,
SEQ ID NO 9:
IRLQISNPDLKDLMVQWFKDGGPSSGAPPPS,
and
SEQ ID NO 10:
PFPPLPIGEEAPKDDMVRFFKDLHQYLNVV.

Another particular embodiment of the present invention relates to CPP-IRF5 peptides as described above, wherein the peptides comprise amino acid sequence SEQ ID NO 13: IRLQISNPYLKFIPLKRAIWLIK.

Another particular embodiment of the present invention relates to CPP-IRF5 peptides as described above, wherein the peptides comprise amino acid sequence SEQ ID NO 14: MIILIISFPKHKDWKVILVK.

Another particular embodiment of the present invention relates to CPP-IRF5 peptides as described above, wherein the peptides comprise amino acid sequence SEQ ID NO 4: MANLGYWLLLLFVTMWTDVGLAKKRPKP.

Another particular embodiment of the present invention relates to CPP-IRF5 peptides as described above, wherein the peptides comprise amino acid sequence SEQ ID NO 5: MANLGYWLALLFVTMWTDVGLFKKRPKP.

Another particular embodiment of the present invention relates to CPP-IRF5 peptides as described above, wherein the peptides comprise amino acid sequence SEQ ID NO 6: MANLGYWLLALFVTYWTDLGLVKKRPKP.

Another particular embodiment of the present invention relates to CPP-IRF5 peptides as described above, wherein the peptides comprise amino acid sequence SEQ ID NO 7: MANLGYWLYALFLTMVTDVGLFKKRPKP.

Another particular embodiment of the present invention relates to CPP-IRF5 peptides as described above, wherein the peptides comprise amino acid sequence SEQ ID NO 8: KDLMVQWFKDGGPSSGAPPPS.

Another particular embodiment of the present invention relates to CPP-IRF5 peptides as described above, wherein the peptides comprise amino acid sequence SEQ ID NO 9: IRLQISNPDLKDLMVQWFKDGGPSSGAPPPS.

Another particular embodiment of the present invention relates to CPP-IRF5 peptides as described above, wherein the peptides comprise amino acid sequence SEQ ID NO 10: PFPPLPIGEEAPKDDMVRFFKDLHQYLNVV.

Another particular embodiment of the present invention relates to pharmaceutical compositions comprising one or more CPP-IRF5 peptides as described above or pharmaceutically acceptable salts thereof and one or more pharmaceutically acceptable excipients.

Another particular embodiment of the present invention relates to CPP-IRF5 peptides as described above or pharmaceutically acceptable salts thereof for the use as therapeutically active substances.

Another particular embodiment of the present invention relates to CPP-IRF5 peptides as described above or pharmaceutically acceptable salts thereof for the use in the treatment or prevention of systemic lupus erythematosus (SLE) or other autoimmune diseases wherein IRF5 signaling plays a significant role.

Another particular embodiment of the present invention relates to a method for the treatment or prevention of systemic lupus erythematosus (SLE) or other autoimmune diseases wherein IRF5 signaling plays a significant role, which method comprises administering CPP-IRF5 peptides as described above or pharmaceutically acceptable salts thereof to a subject.

Another particular embodiment of the present invention relates to the use of CPP-IRF5 peptides as described above or pharmaceutically acceptable salts thereof for the treatment or prevention of systemic lupus erythematosus (SLE) or other autoimmune diseases wherein IRF5 signaling plays a significant role.

Another particular embodiment of the present invention relates to the use of CPP-IRF5 peptides according as described above or pharmaceutically acceptable salts thereof for the preparation of medicaments for the treatment or prevention of systemic lupus erythematosus (SLE) or other autoimmune diseases wherein IRF5 signaling plays a significant role.

The present invention provides compounds to disrupt IRF5 dimerization/signaling and pharmaceutically acceptable salts of such compounds.

In a general embodiment, the compounds are cell-penetrating peptides which bind IRF5 (CPP-IRF5 peptides).

In one embodiment, the compounds are cell-penetrating peptides which bind IRF5 (CPP-IRF5 peptides), wherein the peptide comprises an amino acid sequence selected from the group consisting of SEQ ID NOS: 4-10 and 13-14.

In a particular embodiment, the amino acid sequence comprises at least 20 to about 35 amino acids.

Optionally, the cell-penetrating peptide may also contain or be linked to a small molecule.

In a particular embodiment, the compounds are cell-penetrating peptides which bind interferon regulatory factor IRF5 (CPP-IRF5), wherein the peptide comprises an amino acid sequence of at least 20 to about 35 amino acids, wherein said amino acid sequence further comprises, in part, an amino acid sequence motif selected from the group consisting of

a) Y-R1-R2-R3-L-R4-R5-V (SEQ ID NO: 1),

wherein Y is tyrosine (Tyr), R1 is an amino acid selected from the group of tryptophan (Trp) or alanine (Ala), R2 is an amino acid selected from the group consisting of leucine (Leu) or threonine (Thr), R3 is an amino acid selected from the group consisting of leucine (Leu), alanine (Ala), aspartic acid (Asp) or phenylalanine (Phe), L is leucine (Leu), R4 is an amino acid selected from the group consisting of leucine (Leu), glycine (G) or threonine (Thr), R5 is an amino acid selected from the group consisting of phenylalanine (Phe), leucine (Leu) or methionine (Met), and V is valine (Val); or

b) K-D-R6-M-V-R7-F-K-D (SEQ ID NO: 2),

wherein K is lysine (Lys); D is aspartic acid (Asp), R6 is an amino acid selected from the group consisting of leucine (Leu) or aspartic acid (Asp), M is methionine (Met), R7 is selected from the group consisting of Q-W and R-F, and F is phenylalanine (Phe).

In yet another particular embodiment, the present invention provides an isolated and purified polypeptide of about 8 to about 35 amino acids which binds human interferon regulatory factor IRF5, consisting of a first peptide and an optional second peptide, wherein the first peptide comprises SEQ ID NO: 12 and the second optional second peptide comprising a cell penetrating peptide (CPP) of about 5 to about 20 amino acids. More preferably, polypeptide is SEQ ID. NO: 13 and is cell penetrating.

In an alternative particular embodiment, the present invention provides an isolated and purified polypeptide of about 20 to about 40 amino acids, consisting of a first peptide and an optional second peptide, wherein the first peptide
i. comprises an amino acid sequence of at least 20 amino acids,
ii. has the ability to bind IRF5 and/or inhibit IRF5 dimerization,
iii. and wherein the first peptide further comprises, in part, an amino acid sequence motif of IxLxISxPxxKDxxVxxxK (SEQ ID NO: 15), wherein x is any amino acid,

and the optional second peptide is a cell penetrating peptide (CPP).

In yet another particular embodiment, the present invention provides an isolated and purified peptide of at least 20 to about 40 amino acids, consisting of a first and an optional second polypeptide, wherein the first peptide
i. comprises an amino acid sequence of at least 20 amino acids,
ii. has the ability to bind human interferon regulator factor 5 (IRF5),
iii. and wherein the first peptide comprises, in part, an amino acid motif of K-D-R6-M-V-R7-F-K-D (SEQ ID NO: 2)

and the optional second peptide is a cell penetrating peptide (CPP).

In yet another particular embodiment, the present invention provides SEQ ID NOS. 4-7 and 13-14, which are cell-penetrating peptides which bind human interferon factor 5 (IRF5). Alternatively, the present invention also provides SEQ ID NOS. 8-10 which have the ability to bind interferon regulatory factor 5 (IRF5).

In yet another particular embodiment, the present invention provides an isolated and purified peptide of at least 20 to about 40 amino acids, consisting of a first and an optional second polypeptide, wherein the first peptide
i. comprises an amino acid sequence of at least 20 amino acids,
ii. has the ability to bind human interferon regulator factor 5 (IRF5),
iii. and wherein the first peptide comprises, an amino acid sequence selected from the group consisting of SEQ ID NOS: 8-10

and the optional second peptide is a cell penetrating peptide (CPP).

The present invention also provides a method or assay for screening peptides or small molecules, or a combination or peptide-small molecule, that inhibit IRF5, comprising the following steps:
a) providing a peptide, small molecule or peptide-small molecule to be tested
b) diluting said peptide (or small molecule or peptide-small molecule) in solution
c) preparing a first buffered solution comprising biotin-IRF5 and His-IRF5, wherein each IRF-5 is a mixture of monomer and dimer
d) combining the diluted peptide solution of step b) with the buffered solution of step c) and incubating at room temperature
e) preparing a second buffered solution comprising a fluorescence donor, such as Eu (Europium labeled) conjugated streptavidin, and APC (allophycocyanin) labeled anti-His Ab, as a fluorescence acceptor, for detecting biotin-IRF5 and His-IRF5 dimer formation
f) combining the second buffered solution of step e) with the combined solutions of step d) and incubating at about 4 degrees C. for about 1 day

and determining dimer formation via FRET assay, wherein a decreased FRET signal, as compared to a control group, shows inhibition of IRF5 dimer formation by the peptide (or small molecule or peptide-small molecule) (See e.g. Table 1, FRET assay and IC50 results of SEQ ID NOS: 4-7, 13-14 and 16-21).

More particularly, the IRF5 is selected from the group consisting of mutant S430D (222-467) and White type IRF5 (222-467).

The present invention discloses compounds which are cell-penetrating peptides which bind IRF5 (CPP-IRF5), wherein the peptide comprises an amino acid sequence selected from the group consisting of SEQ ID NOs: 4-10, 13 and 14.

In a particular embodiment, the amino acid sequence comprises at least 20 to about 40 amino acids, more particularly still about at least 20 to about 35 amino acids.

In a particular embodiment, the compounds are cell-penetrating peptides which bind interferon regulatory factor IRF5 (CPP-IRF5), wherein the peptide comprises an amino acid sequence of at least 20 to about 35 amino acids, wherein said amino acid sequence further comprises, in part, an amino acid sequence motif selected from the group consisting of

a) Y-R1-R2-R3-L-R4-R5-V (SEQ ID NO: 1),

wherein Y is tyrosine (Tyr), R1 is an amino acid selected from the group of tryptophan (Trp) or alanine (Ala), R2 is an amino acid selected from the group consisting of leucine (Leu) or threonine (Thr), R3 is an amino acid selected from the group consisting of leucine (Leu), alanine (Ala), aspartic acid (Asp) or phenylalanine (Phe), L is leucine (Leu), R4 is an amino acid selected from the group consisting of leucine (Leu), glycine (G) or threonine (Thr), R5 is an amino acid selected from the group consisting of phenylalanine (Phe), leucine (Leu) or methionine (Met), and V is valine (Val); or

b) K-D-R6-M-V-R7-F-K-D (SEQ ID NO: 2),

wherein K is lysine (Lys); D is aspartic acid (Asp), R6 is an amino acid selected from the group consisting of leucine (Leu) or aspartic acid (Asp), M is methionine (Met), R7 is selected from the group consisting of Q-W and R-F, and F is phenylalanine (Phe).

In a more particular embodiment, the cell-penetrating peptides of the present invention have the amino acid sequence motif of MANLG-Y-R1-R2-R3-L-R4-R5-V(SEQ ID NO: 3). More preferably, the peptide comprises an amino acid sequence selected from the group consisting of SEQ ID NOS 4-7.

In yet another particular embodiment, the present invention provides an isolated and purified peptide of at least 20 to about 40 amino acids, consisting of a first and an optional second polypeptide, wherein the first peptide
i. comprises an amino acid sequence of at least 20 amino acids,
ii. has the ability to bind human interferon regulator factor 5 (IRF5),
iii. and wherein the first peptide comprises, in part, an amino acid motif of K-D-R6-M-V-R7-F-K-D (SEQ ID NO: 2)

and the optional second peptide is a cell penetrating peptide (CPP).

In yet another particular embodiment, the present invention provides SEQ ID NOS. 4-7 and 13-14, which are cell-penetrating peptides which bind human interferon factor 5 (IRF5). Alternatively, the present invention also provides SEQ ID NOS. 8-10 which have the ability to bind interferon regulatory factor 5 (IRF5).

In yet another particular embodiment, the present invention provides an isolated and purified peptide of at least 20 to about 40 amino acids, consisting of a first and an optional second polypeptide, wherein the first peptide
i. comprises an amino acid sequence of at least 20 amino acids,
ii. has the ability to bind human interferon regulator factor 5 (IRF5),
iii. and wherein the first peptide comprises, an amino acid sequence selected from the group consisting of SEQ ID NOS: 8-10

and the optional second peptide is a cell penetrating peptide (CPP).

In yet another particular embodiment, the present invention provides an isolated and purified polypeptide of about 8 to about 35 amino acids which binds human interferon regulatory factor IRF5, consisting of a first peptide and an optional second peptide, wherein the first peptide comprises SEQ ID NO: 12 and the second optional second peptide comprising a cell penetrating peptide (CPP) of about 5 to about 20 amino acids. More preferably, polypeptide is SEQ ID. NO: 13 and is cell penetrating.

In an alternative particular embodiment, the present invention provides an isolated and purified polypeptide of about 20 to about 40 amino acids, consisting of a first peptide and an optional second peptide, wherein the first peptide
i. comprises an amino acid sequence of at least 20 amino acids,
ii. has the ability to bind IRF5 and/or inhibit IRF5 dimerization,
iii. and wherein the first peptide further comprises, in part, an amino acid sequence motif of IxLxISxPxxKDxxVxxxK (SEQ ID NO: 15), wherein x is any amino acid,

and the optional second peptide is a cell-penetrating peptide.

More particularly, the peptide of the present invention consists of the following cell-penetrating peptides:

SEQ ID NO 13:
IRLQISNPYLKFIPLKRAIWLIK
SEQ ID NO 14:
MIILIISFPKHKDWKVILVK
SEQ ID NO 4:
MANLGYWLLLLFVTMWTDVGLAKKRPKP
SEQ ID NO 5:
MANLGYWLALLFVTMWTDVGLFKKRPKP
SEQ ID NO 6:
MANLGYWLLALFVTYWTDLGLVKKRPKP
SEQ ID NO 7:
MANLGYWLYALFLTMVTDVGLFKKRPKP

Alternatively, the peptides of the present invention consist of the following peptides which bind to interferon regulatory factor 5:

SEQ ID NO 8:
KDLMVQWFKDGGPSSGAPPPS
SEQ ID NO 9:
IRLQISNPDLKDLMVQWFKDGGPSSGAPPPS
SEQ ID NO 10:
PFPPLPIGEEAPKDDMVRFFKDLHQYLNVV

The present invention also provides a method or assay for screening peptides or small molecules, or a combination or peptide-small molecule, that inhibit IRF5, comprising the following steps:
a) providing a peptide, small molecule or peptide-small molecule to be tested
b) diluting said peptide (or small molecule or peptide-small molecule) in solution
c) preparing a first buffered solution comprising biotin-IRF5 and His-IRF5, wherein each IRF-5 is a mixture of monomer and dimer
d) combining the diluted peptide solution of step b) with the buffered solution of step c) and incubating at room temperature
e) preparing a second buffered solution comprising a fluorescence donor, such as Eu conjugated streptavidin, and APC (allophycocyanin) labeled anti-His Ab, as a fluorescence acceptor, for detecting biotin-IRF5 and His-IRF5 dimer formation. This assay can be used on any 2 different tag proteins (e.g. GST tag, FLAG tag, HA-tag, Myc-tag, SBP tag or V5 tag). Additionally, any fluorescence donor/acceptor pairs are suitable for use in this assay, as long as the fluorescence emission spectrum of the donor overlaps with the excitation spectrum of the acceptor. Some preferred examples of donor/acceptor dyes are Tb/FITC, Ru/Alexa, FITC/TAMRA and Eu/DyLight. Although the examples below utilize tag proteins for dimer formation and fluorescence conjugated corresponding antibodies or streptavidin for detecting, this assay method can also be performed by labeling proteins directly with donor dyes and acceptor dyes and measure dimer formation by FRET signal. This format can be particularly useful if tagged fusion proteins or antibody binding affect dimer interactions.
f) combining the second buffered solution of step e) with the combined solutions of step d) and incubating at about 4° C. for about 1 day

and determining dimer formation via FRET assay, wherein a decreased FRET signal, as compared to a control group, shows inhibition of IRF5 dimer formation by the peptide (or small molecule or peptide-small molecule) (See, e.g., Table 1, FRET data showing IC50 results).

More particularly, the IRF5 is selected from the group consisting of mutant S430D (222-467) and Wild type IRF5 (222-467).

The compounds of the present invention may be readily synthesized by any known conventional procedure for the formation of a peptide linkage between amino acids. Such conventional procedures include, for example, any solution phase procedure permitting a condensation between the free alpha amino group of an amino acid or fragment thereof having its carboxyl group and other reactive groups protected and the free primary carboxyl group of another amino acid or fragment thereof having its amino group or other reactive groups protected.

Such conventional procedures for synthesizing the novel compounds of the present invention include, for example, any solid phase peptide synthesis method. In such a method the synthesis of the novel compounds can be carried out by sequentially incorporating the desired amino acid residues one at a time into the growing peptide chain according to the general principles of solid phase methods. Such methods are disclosed in, for example, Merrifield, R. B., J. Amer. Chem. Soc. 85, 2149-2154 (1963); Barany et al., The Peptides, Analysis, Synthesis and Biology, Vol. 2, Gross, E. and Meienhofer, J., Eds. Academic Press 1-284 (1980), which are incorporated herein by reference.

During the synthesis of peptides, it may be desired that certain reactive groups on the amino acid, for example, the alpha-amino group, a hydroxyl group, and/or reactive side chain groups, be protected to prevent a chemical reaction therewith. This may be accomplished, for example, by reacting the reactive group with a protecting group which may later be removed. For example, the alpha amino group of an amino acid or fragment thereof may be protected to prevent a chemical reaction therewith while the carboxyl group of that amino acid or fragment thereof reacts with another amino acid or fragment thereof to form a peptide bond. This may be followed by the selective removal of the alpha amino protecting group to allow a subsequent reaction to take place at that site, for example with the carboxyl group of another amino acid or fragment thereof.

Alpha amino groups may, for example, be protected by a suitable protecting group selected from aromatic urethane-type protecting groups, such as allyloxycarbony, benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); and aliphatic urethane-type protecting groups, such as t-butyloxycarbonyl (Boc), diisopropylmethyloxycarbonyl, isopropyloxycarbonyl, and allyloxycarbonyl. In an embodiment, Fmoc is used for alpha amino protection.

Hydroxyl groups (OH) of the amino acids may, for example, be protected by a suitable protecting group selected from benzyl (Bzl), 2,6-dichlorobenztl (2,6 diCl-Bzl), and tert-butyl (t-Bu). In an embodiment wherein a hydroxyl group of tyrosine, serine, or threonine is intended to be protected, t-Bu may, for example, be used.

Epsilon-amino acid groups may, for example, be protected by a suitable protecting group selected from 2-chloro-benzyloxycarbonyl (2-Cl-Z), 2-bromo-benzyloxycarbonyl (2-Br-Z), allycarbonyl and t-butyloxycarbonyl (Boc). In an embodiment wherein an epsilon-amino group of lysine is intended to be protected, Boc may, for example, be used.

Beta- and gamma-amide groups may, for example, be protected by a suitable protecting group selected from 4-methyltrityl (Mtt), 2,4,6-trimethoxybenzyl (Tmob), 4,4′-dimethoxydityl (Dod), bis-(4-methoxyphenyl)-methyl and Trityl (Trt). In an embodiment wherein an amide group of asparagine or glutamine is intended to be protected, Trt may, for example, be used.

Indole groups may, for example, be protected by a suitable protecting group selected from formyl (For), Mesityl-2-sulfonyl (Mts) and t-butyloxycarbonyl (Boc). In an embodiment wherein the indole group of tryptophan is intended to be protected, Boc may, for example, be used.

Imidazole groups may, for example, be protected by a suitable protecting group selected from Benzyl (Bzl), t-butyloxycarbonyl (Boc), and Trityl (Trt). In an embodiment wherein the imidazole group of histidine is intended to be protected, Trt may, for example, be used.

Solid phase synthesis may be commenced from the C-terminal end of the peptide by coupling a protected alpha-amino acid to a suitable resin. Such a starting material can be prepared by attaching an alpha-amino-protected amino acid by an ester linkage to a p-benzyloxybenzyl alcohol (Wang) resin, or by an amide bond between an Fmoc-Linker, such as p-((R,S)-a-(1-(9H-fluoren-9-yl)-methoxyformamido)-2,4-dimethyloxybenzyl)-phenoxyacetic acid (Rink linker), and a benzhydrylamine (BHA) resin. Preparation of the hydroxymethyl resin is well known in the art. Fmoc-Linker-BHA resin supports are commercially available and generally used when the desired peptide being synthesized has an unsubstituted amide at the C-terminus.

In an embodiment, peptide synthesis is microwave assisted. Microwave assisted peptide synthesis is an attractive method for accelerating the solid phase peptide synthesis. This may be performed using Microwave Peptide Synthesizer, for example a Liberty peptide synthesizer (CEM Corporation, Matthews, N.C.). Microwave assisted peptide synthesis allows for methods to be created that control a reaction at a set temperature for a set amount of time. The synthesizer automatically regulates the amount of power delivered to the reaction to keep the temperature at the set point.

Typically, the amino acids or mimetic are coupled onto the Fmoc-Linker-BHA resin using the Fmoc protected form of amino acid or mimetic, with 2-5 equivalents of amino acid and a suitable coupling reagent. After coupling, the resin may be washed and dried under vacuum. Loading of the amino acid onto the resin may be determined by amino acid analysis of an aliquot of Fmoc-amino acid resin or by determination of Fmoc groups by UV analysis. Any unreacted amino groups may be capped by reacting the resin with acetic anhydride and diispropylethylamine in methylene chloride.

The resins are carried through several repetitive cycles to add amino acids sequentially. The alpha amino Fmoc protecting groups are removed under basic conditions. Piperidine, piperazine or morpholine (20-40% v/v) in DMF may be used for this purpose. In an embodiment, 20% piperidine in DMF is utilized.

Following the removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. For example, appropriate reagents for such syntheses are benzotriazol-1-yloxy-tri-(dimethylamino)phosphonium hexafluorophosphate (BOP), bromo-tris-pyrrolidino-phosphonium hexafluorophosphate (PyBroP) 2-(1H-benzotriazole-1-yl)-1,1,3,3-tetramethyluronium hexafluorophosphate (HBTU), and diisopropylcarbodiimide (DIC). In an embodiment, the reagent is HBTU or DIC. Other activating agents are described by Barany and Merrifield (in The Peptides, Vol. 2, J. Meienhofer, ed., Academic Press, 1979, pp 1-284). Various reagents such as 1 hydroxybenzotriazole (HOBT), N-hydroxysuccinimide (HOSu) and 3,4-dihydro-3-hydroxy-4-oxo-1,2,3-benzotriazine (HOOBT) may be added to the coupling mixtures in order to optimize the synthetic cycles. In an embodiment, HOBT is added.

Following synthesis of the peptide, the blocking groups may be removed and the peptide cleaved from the resin. For example, the peptide-resins may be treated with 100 μL ethanedithiol, 100 μl dimethylsulfide, 300 μL anisole, and 9.5 mL trifluoroacetic acid, per gram of resin, at room temperature for 180 min. Alternatively, the peptide-resins may be treated with 1.0 mL triisopropyl silane and 9.5 mL trifluoroacetic acid, per gram of resin, at room temperature for 90 min. The resin may then be filtered off and the peptide precipitated by addition of chilled ethyl ether. The precipitates may then be centrifuged and the ether layer decanted.

Purification of the crude peptide may be, for example, performed on a Shimadzu LC-8A system by high performance liquid chromatography (HPLC) on a reverse phase C18 Column (50×250 mm, 300 Å, 10 μm). The peptides may be dissolved in a minimum amount of water and acetonitrile and injected on to a column. Gradient elution may be generally started at 2%-90% B over 70 minutes, (buffer A: 0.1% TFA/H2O, buffer B: 0.1% TFA/CH3CN) at a flow rate of 60 ml/min. UV detection set at 220/280 nm. The fractions containing the products may be separated and their purity judged on Shimadzu LC-10AT analytical system using reverse phase Pursuit C18 column (4.6×50 mm) at a flow rate of 2.5 ml/min., gradient (2-90%) over 10 min. [buffer A: 0.1% TFA/H2O, buffer B: 0.1% TFA/CH3CN)]. Fractions judged to be of high purity may then be pooled and lyophilized.

Yet another possible method for making the peptides of the present invention would be the following protocol for peptide synthesis at room temperature. In this procedure, generally the following steps would be taken:

| Step | Reagent | Time |
|---|---|---|
| 1 | DMF | 2 × 30 sec |
| 2 | 20% piperidine/DMF | 5 min |
| 3 | 20% piperidine/DMF | 15 min |
| 4 | DMF | 2 × 30 sec |
| 5 | iPrOH | 2 × 30 sec |
| 6 | DMF | 3 × 30 sec |
| 7 | coupling | 60 min-18 hours |
| 8 | DMF | 2 × 30 sec |
| 9 | iPrOH | 1 × 30 sec |
| 10 | DMF | 1 × 30 sec |
| 11 | CH2Cl2 | 2 × 30 sec |

Solvents for all washings and couplings are measured to volumes of 10-20 ml/g resins. Coupling reactions throughout the synthesis can be monitored by the Kaiser Ninhydrin test to determine extent of completion (Kaiser et al. Anal. Biochem. 34, 595-598 (1970)). Any incomplete coupling reactions are either recoupled with freshly prepared activated amino acid or capped by treating the peptide resin with acetic anhydride as described above. The fully assembled peptide-resins are dried in vacuum for several hours, generally overnight, depending on the amount of solvent left.

The amino acid sequences of this invention may also be synthesized by methods known to those of ordinary skill in the art. Such methods include, but are not limited to, microwave peptide synthesis (Murray J. K., Aral J., and Miranda L. P. Solid-Phase Peptide Synthesis Using Microwave Irradiation In Drug Design and Discovery. Methods in Molecular Biology, 2011, Volume 716, 73-88, DOI: 10.1007/978-1-61779-012-6_{—}5) and solid state synthesis of amino acid sequences (Steward and Young, Solid Phase Peptide Synthesis, Freemantle, San Francisco, Calif. (1968)). An exemplary solid state synthesis method is the Merrifield process. Merrifield, Recent Progress in Hormone Res., 23:451 (1967)).}

The compounds of the present invention, as herein described, can also be provided in the form of pharmaceutically acceptable salts. Examples of preferred salts are those formed with pharmaceutically acceptable organic acids, e.g., acetic, lactic, maleic, citric, malic, ascorbic, succinic, benzoic, salicylic, methanesulfonic, toluenesulfonic, trifluoroacetic, or pamoic acid, as well as polymeric acids such as tannic acid or carboxymethyl cellulose, and salts with inorganic acids, such as hydrohalic acids (e.g., hydrochloric acid), sulfuric acid, or phosphoric acid and the like. Any procedure for obtaining a pharmaceutically acceptable salt known to a skilled artisan can be used.

In order to properly dissect the role of IRF5 tool molecules (small molecules or peptides, specifically cell-penetrating peptides) according to this invention, or other suspected small molecules or peptides believed to bind and/or inhibit IRF5, said tool molecules, specifically the cell-penetrating peptides described herein and more specifically in Examples 1-11, a biochemical assay is presented. Due to the lack of a direct approach in the art to biochemically evaluate tools targeting IRF5 dimerization, a novel FRET based biochemical assay of the invention is herein described. The biochemical assay described herein identifies tools that inhibit dimerization of IRF5.

The biochemical FRET assay of the present invention, which provides a method for screening tool molecules (preferably peptides and more preferably cell-penetrating peptides) that inhibit IRF5 by targeting IRF5 (homo)dimerization, generally involves or comprises the following steps:
a) providing a peptide to be tested
b) diluting said peptide in solution
c) preparing a first buffered solution comprising biotin-IRF5 and His-IRF5, wherein each IRF-5 is a mixture of monomer and dimer
d) combining the diluted peptide solution of step b) with the buffered solution of step c) and incubating at room temperature
e) preparing a second buffered solution comprising a fluorescence donor, preferably Eu conjugated streptavidin, and APC (allophycocyanin) labeled anti-His Ab, as a fluorescence acceptor, for detecting biotin-IRF5 and His-IRF5 dimer formation. More generally, the label may be any 2 different tag proteins (e.g. GST tag, FLAG tag, HA-tag, Myc-tag, SBP tag and V5 tag). This assay can be used on any fluorescence donor/acceptor pairs as long as the fluorescence emission spectrum of the donor overlaps with the excitation spectrum of the acceptor. Some preferred examples of donor/acceptor dyes are Tb/FITC, Ru/Alexa, FITC/TAMRA and Eu/DyLight. Although the examples below utilize tag proteins for dimer formation and fluorescence conjugated corresponding antibodies or streptavidin for detecting, this assay method can also be performed by labeling proteins directly with donor dyes and acceptor dyes and measure dimer formation by FRET signal. This format can be particularly useful if tag fusion proteins or antibody binding affect dimer interactions.
f) combining the second buffered solution of step e) with the combined solutions of step d) and incubating at about 4 degrees(4°) C. for about 1 day
g) determining dimer formation via FRET assay, wherein a decreased FRET signal, as compared to a control group, shows inhibition of IRF5 dimer formation by the peptide.

Preferably, the FRET assay is a homogeneous time-resolved fluorescence resonance energy transfer (TR-FRET) assay. More preferably, the IRF5 in step c) is selected from the group consisting of mutant S430D (222-467) and Wild type IRF5 (222-467).

The first buffered solution comprises, in a preferred embodiment, an assay buffer 1 (AB1) consisting of about 20 mM Hepes, 100 mM NaCl, 0.1 mM EDTA, 1 mM DTT, 0.2 mg/ml BSA, at an pH of about 7.0.

In a preferred embodiment of the method of the invention, the testing peptide solutions are serially diluted 2-3 fold (approximately 2 mM) in DMSO and are transferred 2.5 ul/well of each solution into 96-well polypropylene (PP) plate. The following steps may then be performed:
1) Prepare 100 nM of biotin-IRF5(S430D) (0.96 mg/ml or 32 uM,) and 250 nM His-IRF5(S430D) (1.51 mg/ml or 51 uM,) in AB1
2) Add 50 ul/well of solution in (1) into peptide solutions in in 96-well PP plates as described above and incubate at RT for 20 min.
3) Prepare 10 nM Eu-labeled streptavidin and 80 nM APC-labeled anti-His Ab in AB2 (AB1 w/o DTT) containing 5% DMSO and add 17 ul/well into solutions in (4).
4) Transfer 30 ul/well into 384-well PP plate (Matrix) and incubate at 40 C for 1 day.

The FRET assay is then performed and read, for example on Envision, with excitation at 340 nm and emission at 615 nm (donor fluorescence) and 665 nm (acceptor fluorescence) to determine the FRET signal, wherein a decreased FRET signal, as compared to a control group, shows inhibition of IRF5 dimer formation by the peptide.

More specific examples of the assay are exemplified below in Examples 12-13. These examples however do not limit the scope of the method invention described herein.

All publications, patent applications, patents, and other references mentioned herein are incorporated by reference in their entirety.

Pharmaceutical Compositions

In another aspect the invention provides a pharmaceutical composition comprising cell-penetrating peptides which bind interferon regulatory factor IRF5 (CPP-IRF5 peptides), in a pharmaceutically acceptable carrier. These pharmaceutical compositions may be used, e.g., in any of the therapeutic methods described below.

Pharmaceutical compositions of CPP-IRF5 peptides as described herein are prepared by mixing such CPP-IRF5 peptides having the desired degree of purity with one or more optional pharmaceutically acceptable carriers (Remington's Pharmaceutical Sciences 18th edition, Mack Printing Company (1990)), in the form of lyophilized formulations or aqueous solutions. Pharmaceutically acceptable carriers are generally non-toxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH2O, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The pharmaceutical composition herein may also contain additional active ingredients as necessary for the particular indication being treated, particularly those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, Mack Printing Company (1990).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The compositions to be used for in vivo administration are generally sterile. Sterility may be readily accomplished, e.g., by filtration through sterile filtration membranes.

### EXAMPLES

The invention will be more fully understood by reference to the following examples. They should not, however, be construed as limiting the scope of the invention.

### Example 1

Peptides with SEQ ID NO 4-7 and 13-14 were synthesized [by CSBio (Menlo Park, Calif., USA)] via solid state synthesis. (Steward and Young, Solid Phase Peptide Synthesis, Freemantle, San Francisco, Calif. (1968). The general exemplary method for the solid state synthesis for said sequences is described as follows:

Material:

All chemicals and solvents such as DMF (Dimethylformamide), DCM (Methylene Chloride), DIEA (Diisopropylethylamine), and piperidine were purchased from VWR and Aldrich, and used as purchased without further purification. Mass spectra were recorded with Electrospray ionization mode. The automated stepwise assembly of protected amino acids was constructed on a CS 336X series peptide synthesizer (C S Bio Company, Menlo Park, Calif., USA) with Rink Amide MBHA resin as the polymer support. N-(9-fluorenyl)methoxycarbonyl (Fmoc) chemistry was employed for the synthesis. The protecting groups for Fmoc amino acids (AAs) were as follows, Arg: (Pbf), Asn/Gln/Cys/His: (Trt), Asp/Glu: (OtBu), Lys/Trp: (Boc), Ser/Thr/Tyr: (tBu).

Synthesis:

In general, the synthesis route started from deFmoc of pre-loaded Rink Amide resin and coupling/de-protecting of desired AAs according to the given sequences for all the orders. Coupling reagent was DIC/HOBt, and reaction solvents were DMF and DCM. The ratio of peptidyl resin/AA/DIC/HOBT was 1/4/4/4 (mol/mol). After coupling program, DeFmoc was executed using 20% piperidine in DMF. For example, a 0.4 mmol synthesis was performed till the last AA was attached. After deFmoc, the resin was acetylated with Ac2O/DIEA to give N-term Ac sequence or cleaved from the resin without acetylation to give N-Term Amine sequence.

Fmoc-Rink Amide Resin (0.85 g, 0.4 mmol, sub: 0.47 mm/g, Lot#110810, C S Bio) was mixed in a 25 mL reaction vessel (RV) with DMF (10 mL), and swollen for 10-30 min. The RV was mounted on a CS336 peptide automated synthesizer and the amino acids were loaded onto amino acid (AA) wheel according to the given peptide sequence. HOBt (0.5M in DMF) and DIC (0.5M in DMF) were all pre-dissolved separately in transferrable bottles under N2. Fmoc-amino acids (AAs, 4 eq) were weighed and prelocated as powder on the AA wheel. For example, 0.4 mmol synthesis needed 1.6 mmol of AA. The preset program started from AA dissolving in the AA tube and the solution was pumped thru M-VA to T-VA. HOBt solution was later mixed with AA. N2 bubbling was used to assist mixing. While DIC solution was combined with the AA/HOBt solution, the whole mixture was transferred into the RV with drained resin in 5 min and the coupling started at the same time. After shaking for 3-6 hr, reaction mixture was filtered off and the resin was washed with DMF three times, followed by deFmoc according to the preset program using 20% Pip in DMF. The next AA was attached following the same route. Seven washing steps were done with DMF/DCM alternatively after deFmoc. The coupling process was repeated with the respective building blocks according to the given sequence till the last AA was coupled. Coupling Time: 3-6 hrs for each AA attachment.

After deFmoc of last AA, the resin was acetylated by Ac2O/DIEA in DMF or cleaved from the resin without acetylation to give N-Term Amine sequence.

Cleavage:

The final peptidyl resin (1-1.5 g) was mixed with TFA cocktail (TFA/EDT/TIS/H2O) and the mixture was shaken at room temperature for 4 hr. The cleaved peptide was filtered and the resin was washed by TFA. After ether precipitation and washing, the crude peptide (200-500 mg) was obtained in a yield of 50-90%. The crude peptide was directly purified without lyophilization.

Purification:

Crude peptides, 200-500 mg of acetylated or non-acetylated peptides, were dissolved in Buffer A 0.1% TFA in water and ACN, and the peptide solution was loaded onto a C18 column (2 inch) with a prep HPLC purification system. With a flow rate of 25-40 mL/min, the purification was finished in a TFA (0.1%) buffer system with a 60 min gradient. Fractions (peptide purity>95%) containing the expected MW were collected. The prep HPLC column was then washed for at least three void column volumes by 80% Buffer B and equilibrated to 5% Buffer B before next loading.

Lyophilization:

The fractions (purity>90%) were combined and transferred to 1 L lyophilization jars which were deeply frozen by liquid nitrogen. After freezing, the jars were placed onto Lyophilizer (Virtis Freezemobile 35EL) and dried overnight. The vacuum was below 500 mT and chamber temperature was below −60° C. The lyophilization was completed in 12-18 hrs at room temperature (environment temperature).

Results:

In starting 0.4 mm synthesis for each sequence, the synthesis yield was around 50-90% and the crude purities ranged from 30-70%. The purification was done in TFA system and final yield was about 10% for each order.

### Example 2

### Synthesis of Ac-IRLQISNPYLKFIPLKRAIWLIK-NH₂ (SEQ ID NO: 13)

The above peptide was synthesized [by CSBio (Menlo Park, Calif., USA)] as per Example 1 above via solid state synthesis. In the specific preparation of SEQ ID NO:13, Fmoc Rink Amide MBHA resin was subjected to solid phase synthesis and purification by following the procedure in example 1 to yield 125 mg (yield: 10.2%; purity: 96.9%). (ES)+-LCMS m/e calculated (“calcd”) for C140H230N36O28. found 2865.20.

### Example 3

### Synthesis of Ac-MIILIISFPKHKDWKVILVK-NH₂ (SEQ ID NO: 14)

The above peptide was synthesized [by CSBio (Menlo Park, Calif., USA)] as per Example 1 above via solid state synthesis. In the specific preparation of SEQ ID NO:14, Fmoc Rink Amide MBHA resin was subjected to solid phase synthesis and purification by following the procedure in example 5 to yield 118 mg (yield: 4.8%; purity: 97.4%). (ES)+-LCMS m/e calculated (“calcd”) for C121H200N28O24S. found 2463.06.

### Example 4

### Synthesis of Ac-MANLGYWLLLLFVTMWTDVGLAKKRPKP-NH₂ (SEQ ID NO: 4)

The above peptide was synthesized [by CSBio (Menlo Park, Calif., USA)] as per Example 1 above via solid state synthesis. In the specific preparation of SEQ ID NO:4, Fmoc Rink Amide MBHA resin was subjected to solid phase synthesis and purification by following the procedure in example 5 to yield 145 mg (yield: 9.4%; purity: 95.4%). (ES)+-LCMS m/e calculated (“calcd”) for C156H245N37O3552. found 3262.66.

### Example 5

### Synthesis of Ac-MANLGYWLALLFVTMWTDVGLFKKRPKP-NH₂ (SEQ ID NO: 5)

The above peptide was synthesized [by CSBio (Menlo Park, Calif., USA)] as per Example 1 above via solid state synthesis. In the specific preparation of SEQ ID NO:5, Fmoc Rink Amide MBHA resin was subjected to solid phase synthesis and purification by following the procedure in example 5 to yield 116 mg (yield: 7.0%; purity: 96.4%). (ES)+-LCMS m/e calculated (“calcd”) for C159H243N37035 S2 found 3296.40

### Example 6

### Synthesis of Ac-MANLGYWLLALFVTYWTDLGLVKKRPKP-NH₂ (SEQ ID NO: 6)

The above peptide was synthesized [by CSBio (Menlo Park, Calif., USA)] as per Example 1 above via solid state synthesis. In the specific preparation of SEQ ID NO:6, Fmoc Rink Amide MBHA resin was subjected to solid phase synthesis and purification by following the procedure in example 5 to yield 210 mg (yield: 14.7%; purity>:97.7%). (ES)+-LCMS m/e calculated (“calcd”) for C160H245N37O36S. found 3294.40

### Example 7

### Synthesis of Ac-MANLGYWLYALFLTMVTDVGLFKKRPKP-NH₂ (SEQ ID NO: 7)

The above peptide was synthesized [by CSBio (Menlo Park, Calif., USA)] as per Example 1 above via solid state synthesis. In the specific preparation of SEQ ID NO:7, Fmoc Rink Amide MBHA resin was subjected to solid phase synthesis and purification by following the procedure in example 5 to yield 189 mg (yield: 5.8%; purity: >96%). (ES)+-LCMS m/e calculated (“calcd”) for C157H242N36O36S2. found 3274.26

### Example 8

Peptides with SEQ ID NOS 8-10 were synthesized by HYBio (Shenzhen, China) via solid state synthesis. The general exemplary method for the synthesis for said sequences is described as follows.

Peptides of SEQ ID NOS 8-10 were synthesized using Fmoc chemistry. The synthesis was carried out on a 0.15 mmole scale using the Fmoc-Linker-Rink amide resin (0.5 g, Sub=0.3 mmol/g). 0.5 g of dry resin was placed in a peptide synthesis reactor column (20×150 mm), swollen and washed with DMF, followed by addition of 20% piperidine, agitation for 5 min, draining, addition of 20% piperidine, agitation for 7 min, resin wash with DMF. 0.75 mmol (5 eq) Fmoc-Arg(Pbf)-OH, 0.75 mmolHOBt [symbol] 0.75 mmol HBTU, and 0.75 mmol DIPEA were added into the reaction column, followed by gentle agitation for 2 hours with Nitrogen. Some resin sample was taken for color test, and after that the Fmoc group was deprotected. The steps above were repeated until all the amino acids were coupled. At the end of the synthesis, the resin was transferred to a reaction vessel on a shaker for cleavage. The peptide was cleaved from the resin using 20.0 mL cleavage cocktail (TFA:TIS:H2O:EDT=91:3:3:3(v/v)) for 120 minutes at room temperature avoiding light. The deprotection solution was added to 1000 mL cold Et2O to precipitate the peptide. The peptide was centrifuged in 250 mL polypropylene tubes. The precipitates from the individual tubes were combined in a single tube and washed three times with cold Et20 and dried in a desiccator under house vacuum.

The crude material was purified by preparative HPLC on a C18-Column (250×46 mm, 10 μm particle size) and eluted with a linear gradient of 5-95% B (buffer A: 0.1% TFA/H2O; buffer B:ACN) in 30 min., with a flow rate of 19 mL/min, and detection 220 nm. The fractions were collected and were checked by analytical HPLC. Fractions containing pure product were combined and lyophilized to a white amorphous powder.

### Example 9

### Synthesis of Ac-KDLMVQWFKDGGPSSGAPPPS-NH₂ (SEQ ID NO: 8)

The above peptide was synthesized [by Hybio (Shenzhen, China)] as per Example 8 above via solid state synthesis. In the specific preparation of SEQ ID NO:8, Fmoc-Linker-Rink amide resin was subjected to solid phase synthesis and purification by following the procedure in example 8 (yield: 20%; purity: >95%). (ES)+-LCMS m/e calculated (“calcd”) for C101H152N2603051 found 2242.56

### Example 10

### Synthesis of Ac-IRLQISNPDLKDLMVQWFKDGGPSSGAPPPS-NH₂ (SEQ ID NO: 9)

The above peptide was synthesized [by Hybio (Shenzhen, China)] as per Example 8 above via solid state synthesis. In the specific preparation of SEQ ID NO:9, Fmoc-Linker-Rink amide resin was subjected to solid phase synthesis and purification by following the procedure in example 8 (yield: 20%; purity: >95%). (ES)+-LCMS m/e calculated (“calcd”) for C152H239N41O45S1. found 3392.91

### Example 11

### Synthesis of Ac-PFPPLPIGEEAPKDDMVRFFKDLHQYLNVV-NH₂ (SEQ ID NO: 10)

The above peptide was synthesized [by Hybio (Shenzhen, China)] as per Example 8 above via solid state synthesis. In the specific preparation of SEQ ID NO:10, Fmoc-Linker-Rink amide resin was subjected to solid phase synthesis and purification by following the procedure in example 8 (yield: 20%; purity: >95%). (ES)+-LCMS m/e calculated (“calcd”) for C167H250N40O44S1. found 3554.16

In order to properly dissect the role of IRF5 tool molecules (small molecules or peptides) according to this invention, or other suspected small molecules or peptides believed to bind and/or inhibit IRF5, said tool molecules, specifically the cell-penetrating peptides described above in Examples 2-11, a biochemical assay would be needed. Due to the lack of a direct approach in the art to biochemically evaluate tools targeting IRF5 dimerization, a novel FRET based biochemical assay was established. The biochemical assay described herein identifies tools that inhibit dimerization of IRF5. Generally, the synthesized peptides described in Examples 2-11 above were first tested in biochemical assays (FRET) and then further evaluated in cell based assays. The first cell based assay used was TLR7/8 ligand (R848) stimulated IL6 production in THP1 cells, a system that we confirmed to be dependent on IRF5 using an siRNA approach. Selectivity of the compounds was measured in an NFkB translocation assay and cytotoxicity was measured using assays described herein. Our data show that we have developed novel tools that allow us to determine if the tools/peptides block IRF5 homo-dimerization in a biochemical assay as well as interrogate IRF5 function in vitro.

### Example 12

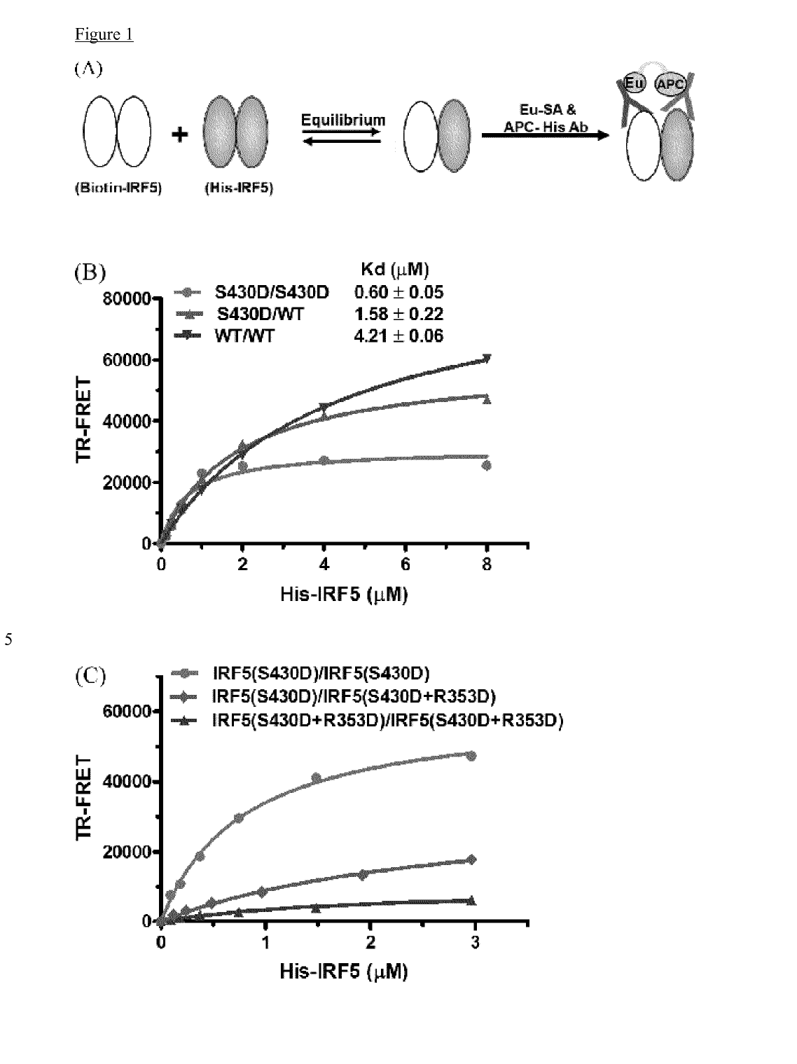

### FIG. 1

### IRF5 Dimerization Assay

The dimerization of IRF5 has been reported to be critical to IRF5 nuclear translocation and function. To test the ability of compounds to inhibit IRF5 dimerization a time-resolved fluorescence resonance energy transfer (TR-FRET) was developed. Binding of recombinant His tagged IRF5 (222-467 constructs) to recombinant biotin tagged IRF5 was measured by FRET between Europium labeled anti-GST antibody and Stretavidin-conjugated Allophycocyanin. The ability of IRF5 constructs to dimerize was first determined using multiple constructs (FIG. 1). The IRF5 S430 and WT (222-467) constructs were then used to test the ability of compounds to inhibit dimerization. Typically, test peptides (2 mM stock in DMSO) were diluted 3 fold in series in DMSO and 2.5 ul per well were added into 96-well polypropylene plates (Corning). Fifty microliters per well of 100 nM biotin tag IRF5(222-467, S430D) and 250 nM His tag IRF5(222-467, S430D) in Assay Buffer (50 mM Tris-HCl, pH 7.4, 100 mM NaCl, 1 mM DTT and 0.2 mg/ml BSA) were added. The samples were incubated at room temperature for 20 min. Seventeen microliters per well of detection solution containing 10 nM europium (Eu) conjugated streptavidin and 80 nM allophycocyanin (APC) conjugated anti-His antibody (Columbia Biosciences) in Assay Buffer (without DTT) were added. The samples were incubated at room temperature for 60 min followed by overnight incubation at 40 C and 30 ul per well were transferred to 384-well polystyrene plates (Matrix, Thermal Scientific) in duplicates. Assay signals were monitored by reading excitation at 340 nm and emission fluorescence at 615 nm and 665 nm on an Envision reader. TR-FRET signals were calculated by the ratio of acceptor to donor signals after subtracting both blank and donor cross-talk values from acceptor signal (Huang, K S and Vassilev, L T, Methods Enzymol., 399, 717-728 (2005)). The data were processed in Excel XLfit and the IC50 values were calculated using a nonlinear curve-fitting algorithm (four parameter equation). The data represent an average of 3 independent experiments (each run in triplicates) and the reported errors represent standard deviation (s.d.). This assay can be used on any 2 different tag proteins (e.g. GST tag, FLAG tag, HA-tag, Myc-tag, SBP tag and V5 tag)

This assay can be used on any fluorescence donor/acceptor pairs as long as the fluorescence emission spectrum of the donor overlaps with the excitation spectrum of the acceptor. Some of examples of donor/acceptor dyes are Tb/FITC, Ru/Alexa, FITC/TAMRA and Eu/DyLight

Although we have shown examples using tag proteins for dimer formation and fluorescence conjugated corresponding antibodies for detecting, this assay can also be performed by labeling proteins directly with donor dyes and acceptor dyes and measure dimer formation by FRET signal. This format can be particularly useful if tag fusion proteins or antibody binding affect dimer interactions.

### Example 13

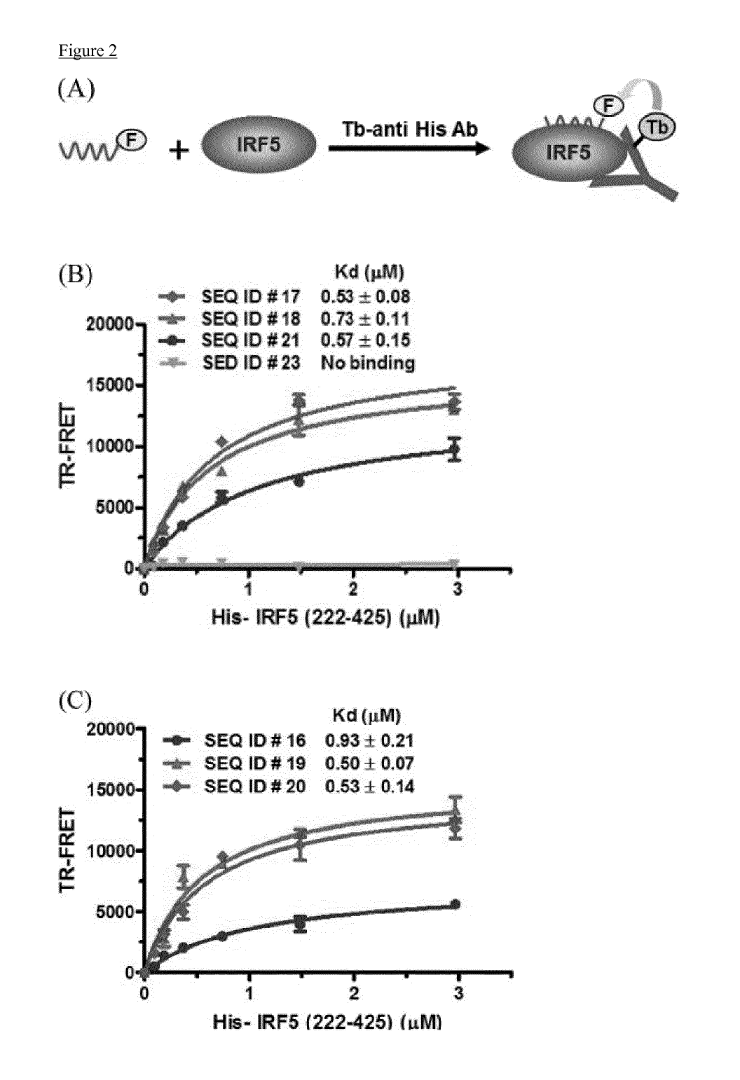

### FIG. 2

### Kd Determination of FITC Peptide Binding to IRF5

The ability of FITC tagged CPP's SEQ ID NOS 4-7 and 13-14 (FITC labeled versions of CPP's SEQ ID NOS: 4-7 and 13-14 are listed as SEQ ID NOS: 16-21) to directly bind IRF5 was tested using a modified TR-FRET assay. Binding of the FITC CPP to His tagged recombinant IRF5 was measured by FRET between FITC and Terbium tagged anti-His antibody. Aliquots (1.6 μl per well) of 4 μM FITC peptide solution in DMSO were added into 96-well polypropylene plates (Corning). Thirty microliters (30 μl) per well of various concentrations (0-10.5 uM, 2 fold serial dilution) of His tag IRF5(222-425) in Assay Buffer (50 mM Tris-HCl, pH 7.4, 100 mM NaCl, 1 mM DTT and 0.2 mg/ml BSA) were added into FITC peptide containing wells. The samples were incubated at room temperature for 30 min. Ten microliters (10 μl) per well of different concentrations of Tb labeled anti His antibody in Assay Buffer (without DTT) were added into wells containing corresponding concentrations of IRF5 solution to keep the same ratio of IRF5 to Tb (10 to 1). Samples were incubated at 4° C. for overnight and 18 μl per well were transferred to small volume 384-well polystyrene plates (Corning) in duplicates. Assay signals were monitored by reading excitation at 340 nm and emission fluorescence at 495 nm and 525 nm on an Envision reader. The TR-FRET signals were calculated from the fluorescence intensities at 525 nm after subtracting the background from assay buffer. The data were processed in Prism software (GraphPad) and the Kd values were calculated using one-site specific binding algorithm. The data represent an average of 3 experiments (each in triplicates) and the reported errors represent s.d.

### Example 14

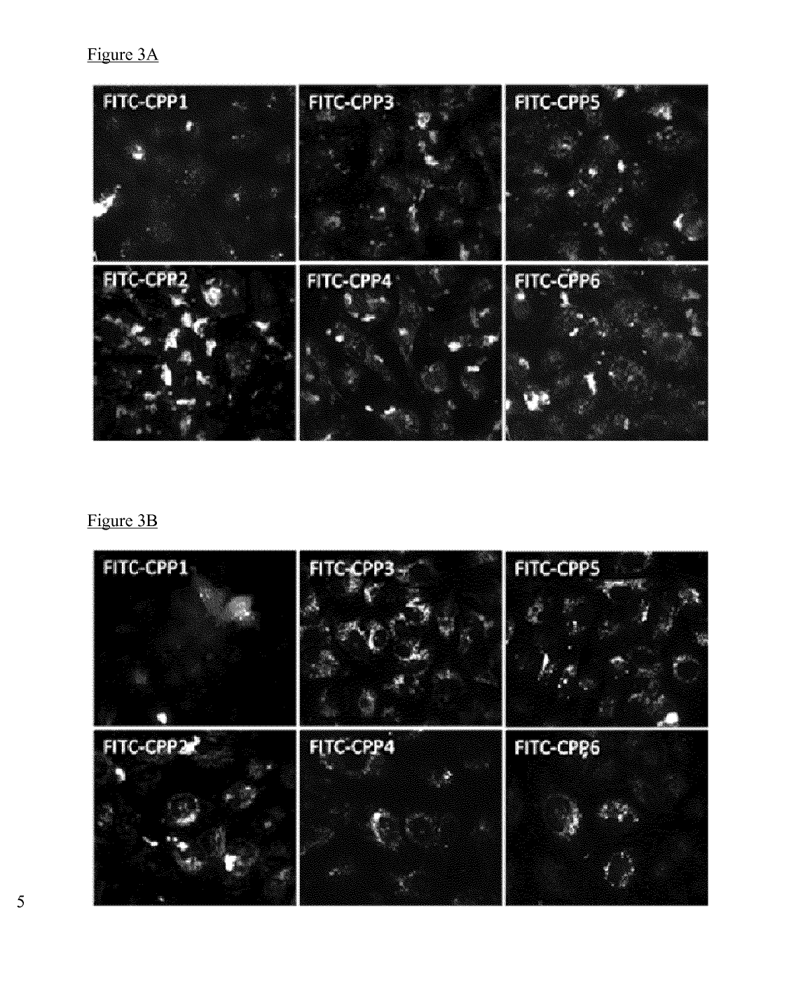

### FIG. 3

### Cell Penetration of FITC Tagged CPPs SEQ ID NOS 16-21

The ability of FITC tagged CPPs to penetrate cells was determined by confocal microscopy. HeLa cells, 5 k/well were plated onto Whatman glass-bottom 96-well plates for FITC uptake analysis at 2 hr. and 24 hr. The next day peptides were added at various concentrations in complete media (RPMI, 10% serum). 2 h and 24 h after addition of peptides media was removed and cells were washed three times with 504/well acidic saline (pH 3) and fixed with 37′C fixative (19.9 mL Hanks/HEPES per 2.2 mL formaldehyde) for 15 min followed by a two rinses in PBS. Cellular uptake of the FITC-labeled peptides SEQ ID NOS 16-21 was assessed by automated confocal microscopy and images were obtained at 40× magnification.

### Example 15

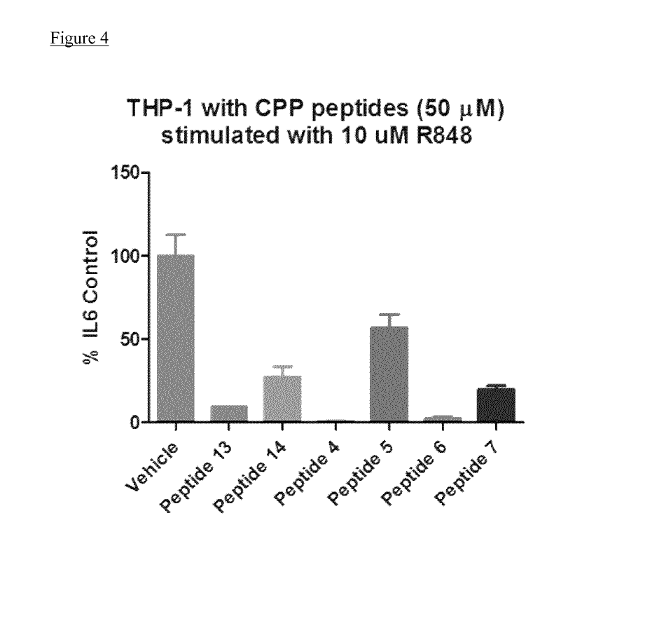

### FIG. 4

THP-1 cells obtained from ATCC were seeded at 50 k cells/100 μl/well in a 96 well plate (Corning Cat#3340). Peptides were dissolved in DMSO at 10 mM as stock solution, then 1:10 in water at 1 mM, mix well. R848 (Enzo Cat#ALX-420-038-M005) was dissolved in DMSO (Sigma Cat#D2650) at 10 mM. 5 μl of CPP stock (1 mM) was added to 96-well cell plate, the final concentration of CPP is 50 μM, and then incubated 30 minutes at 37° C. R848 was added to the 96-well cell plate at a final concentration of 10 μM, and cells were incubated at 37° C. for 24 h. The supernatant was tested for IL6 by Alphalisa (Perkin Elmer AL233C) as per manufacturer's instructions. Viability of the cells was measured by cell titer glo (Promega).

### Example 16

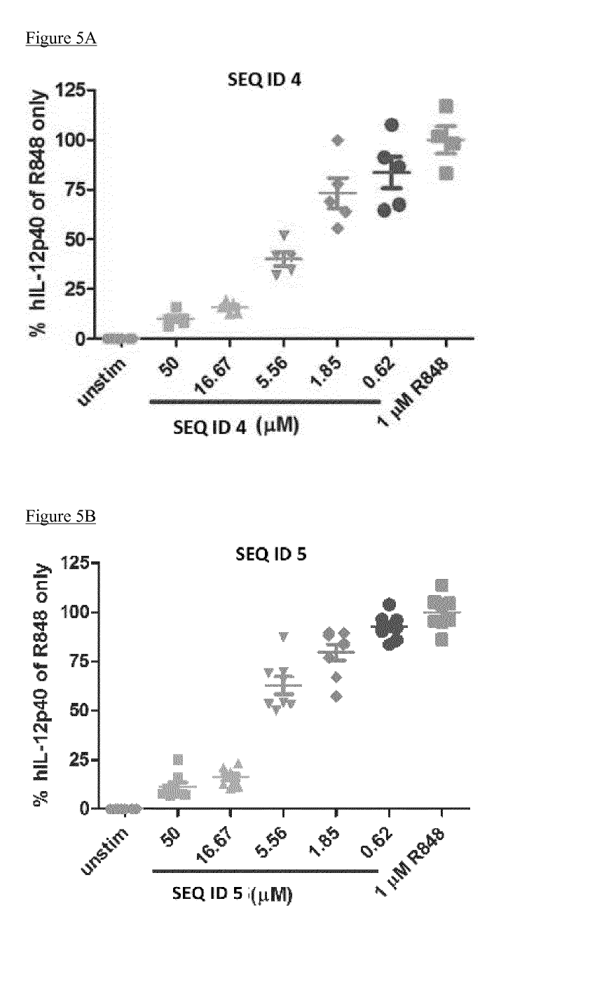

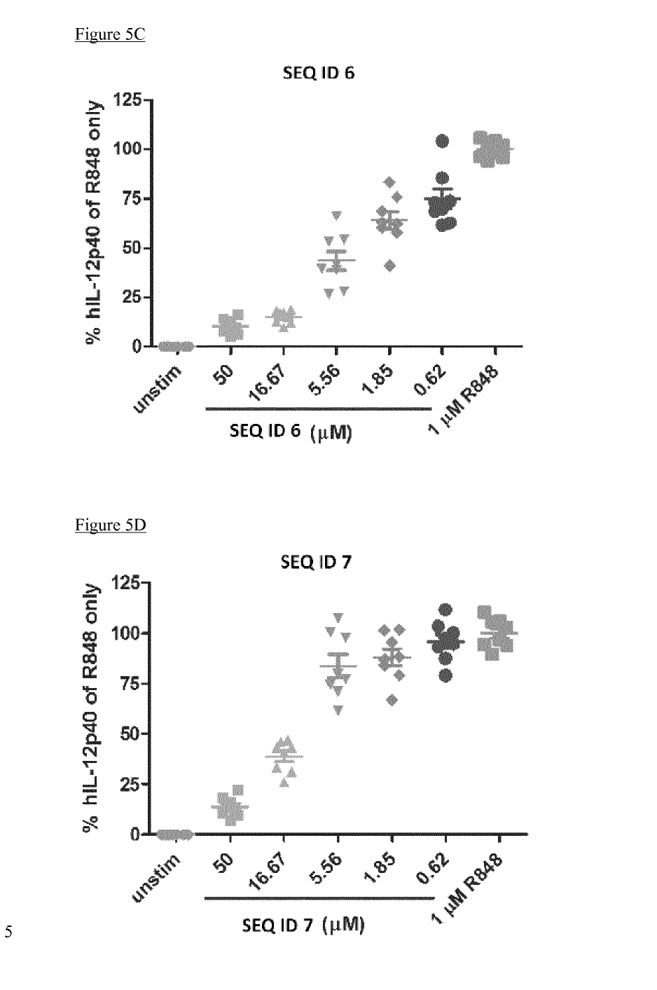

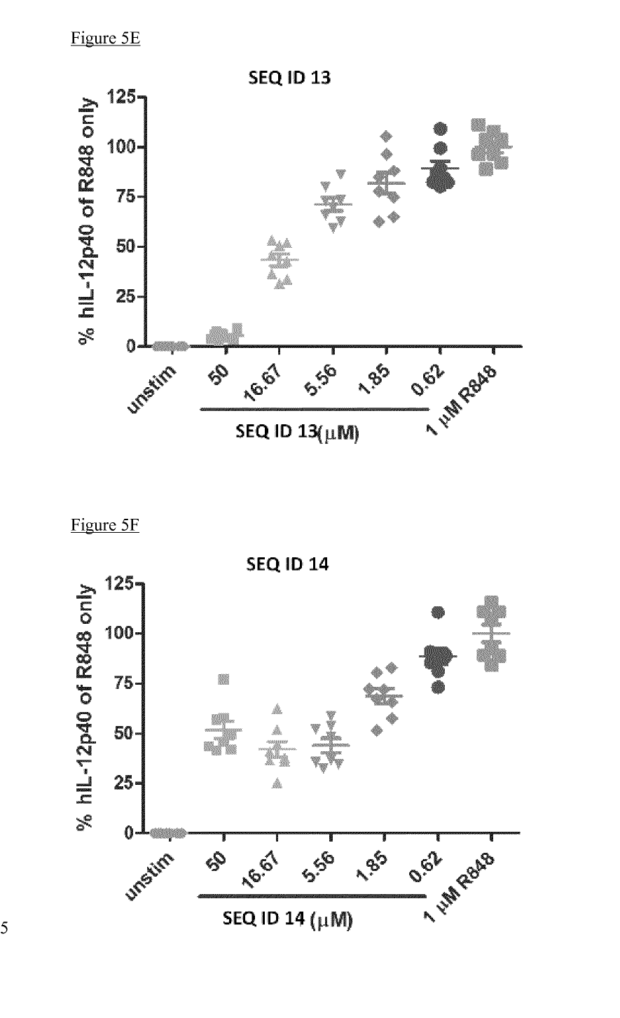

### FIGS. 5a-5f

Human peripheral blood mononuclear cells (PBMC) were isolated from healthy volunteer blood (using protocol and guidelines approved by IRB) using Ficoll density based separation. Purified PBMC's were seeded at 100 k cells/well in 96-well cell-culture compatible plates. The cells were pre-treated with various concentrations of peptides for 30 min at 37° C. and stimulated with 1 μM R848 o/n at 37° C. The R848 stimulated secretion of human IL-12p40 was measured using ELISA (BD (Becton Dickinson), cat#555171) according to manufacturer's instructions.

### Example 17

### NFκB Translocation Assay Protocol (Results Shown in Table 3)

The selectivity of the CPP's over NFκB was determined using a high content screening assay wherein TNFα mediated nuclear translocation of NFκB was determined by imaging.

HeLa cells were plated at 5000 cells/well in 96 well Perkin Elmer ViewPlates and incubated overnight at 37 C. Media was aspirated and compounds pre-diluted in in 0.05% BSA Hanks/20 mM HEPES were added in duplicate at various concentrations for 30 minutes. Wells were stimulated with 20 μl of 150 ng/ml TNFα for 30 minutes at 37 C. Wells were aspirated and the cells were fixed with 3.7% formaldehyde solution for 15 minutes at room temperature. The fixative was removed and the plates were washed with PBS. The NFkB translocation assay, based upon detection of an antibody to p65 (Thermo-Fisher), was completed and read on the Perkin Elmer Operetta at 40×.

Cell Titer-Glo Assay Protocol

The toxicity of the peptides was determined by measuring cellular ATP content as a surrogate for cell number. Briefly, HeLa 3000 cells/well in 96 well Perkin Elmer ViewPlates and incubated overnight at 37° C. Media was aspirated and compounds pre-diluted in growth media were added in duplicate at various concentrations for 24 hours. Cell Titer-glo reagent (Promega) was added to each well as per the protocol provided. The cells were placed on a shaker for 2 minutes and incubated for an additional 10 minutes at room temperature. The plates were read on the Perkin Elmer Envision plate reader for luminescence.

TABLE 1
Potencies of CPP-IRF5 in FRET IRF5 dimerization
inhibition assay

| Seq. | | S430D | WT |
|---|---|---|---|
| ID | Sequence | IC₅₀ (μM) | IC₅₀ (μM) |
| 13 | Ac-IRLQISNPYLKFIPLK | >75 | >75 |
| | RAIWLIK-NH₂ | | |
| 14 | Ac-MIILIISFPKHKDWKV | 35.1 ± 4.7 | 18.6 ± 3.3 |
| | ILVK-NH₂ | | |
| 4 | MANLGYWLLLLFVTMWTDV | 26.4 ± 4.5 | 19.5 ± 4.0 |
| | GLAKKRPKP | | |
| 5 | MANLGYWLALLFVTMWTDV | 26.6 ± 5.4 | 22.4 ± 6.2 |
| | GLFKKRPKP | | |
| 6 | MANLGYWLLALFVTYWTDL | 15.4 ± 5.8 | 15.3 ± 4.8 |
| | GLVKKRPKP | | |
| 7 | MANLGYWLYALFLTMVTDV | 34.2 ± 5.5 | 41.9 ± 6.4 |
| | GLFKKRPKP | | |
| 16 | FITC-Aha-IRLQISNPYL | >61.6 | 46.5 ± 10.1 |
| | KFIPLKRAIWLIK-NH₂ | | |
| 17 | FITC-Aha-MIILIISFPK | 10.7 ± 3.2 | 5.3 ± 2.5 |
| | HKDWKVILVK-NH₂ | | |
| 18 | FITC-Aha-MANLGYWLLL | 8.5 ± 3.0 | 2.9 ± 0.5 |
| | LFVTMWTDVGLAKKRPKP | | |
| 19 | FITC-Aha-MANLGYWLAL | 8.9 ± 2.6 | 2.3 ± 0.4 |
| | LFVTMWTDVGLFKKRPKP | | |
| 20 | FITC-Aha-MANLGYWLLA | 8.9 ± 2.8 | 2.9 ± 0.3 |
| | LFVTYWTDLGLVKKRPKP | | |
| 21 | FITC-Aha-MANLGYWLYA | 10.9 ± 3.4 | 3.5 ± 0.6 |
| | LFLTMVTDVGLFKKRPKP | | |
| 23 | Ac-YLKFIPLKRAIWLIK- | >75 | >75 |
| | NH₂ | | |

This table displays the potency (IC50 in μM, columns 3 and 4) of the CPP's 13-14 and 4-7 (SEQ ID NOS: 13-14 and 4-7) and their FITC labeled versions (SEQ ID NOS: 16-21) in the FRET assay described in example 12. The FRET assay was performed using S430D phosphomimetic construct of IRF5 (222-467) as well as WT (222-467). A control CPP designed not to bind IRF5 (SEQ ID NO: 23) does not display any affinity.

TABLE 2
Potencies of CPP-IRF5 (SEQ ID NOS: 8-10) in FRET
IRF5 dimerization inhibition assay

| Sequence | | S430D | WT IC₅₀ |
|---|---|---|---|
| ID | Sequence | IC₅₀ (μM) | (μM) |
| 8 | Ac-KDLMVQWFKDGGPSSGAPPPS- | 1.68 | 0.79 |
| | NH₂ | | |
| 9 | Ac-IRLQISNPDLKDLMVQWFKDGG | 2.42 | 1.4 |
| | PSSGAPPPS-NH₂ | | |
| 10 | Ac-PFPPLPIGEEAPKDDMVRFFKD | 1.67 | 0.35 |
| | LHQYLNVV-NH₂ | | |

This table displays the potency (IC50 in μM, columns 3 and 4) of SEQ ID NOS: 8-10 in the FRET assay described in example 12. The FRET assay was performed using S430D phosphomimetic construct of IRF5 (222-467) as well as WT (222-467), according to the procedure of Example 12.

TABLE 3
SEQ ID NOS: 13-14 and 4-7 are selective and not cytotoxic

| Sequence | NFkB selectivity | Cytotoxicity in THP-1 cells |
|---|---|---|
| ID | (50 μM) | at 10 μM |
| 13 | No inhibition | None |
| 14 | No inhibition | None |
| 4 | No inhibition | None |
| 5 | No inhibition | None |
| 6 | No inhibition | None |
| 7 | No inhibition | None |

The data with the SEQ ID NOS: 13-14 and 4-7 in the NFkB selectivity assay and cytotoxicity assay (cell titer glo, Promega) are summarized in this table. The CPP's tested did not significantly attenuate TNFα induced NFkB translocation in HeLa cells establishing specificity for IRF5 over NFkB. In addition, the CPP's tested were not cytotoxic (wherein cytotoxicity is defined as greater than 40% loss of cells) in HeLa cells after 24 h incubation with the peptides.

1. A cell-penetrating peptide which binds interferon regulatory factor IRF5 (CPP-IRF5), wherein the peptide comprises an amino acid sequence of 20 to 40 amino acids and wherein said amino acid sequence further comprises, in part, an amino acid sequence motif selected from the group consisting of:
a) I-x-L-x-I-S-x-P-x-x-K (SEQ ID NO: 30), wherein
I isisoleucine,
L is leucine,
S is serine,
P is proline,
K is lysine, and
x is independently selected from any amino acid; or
b) Y-R1-R2-R3-R8-R4-R5-R9 (SEQ ID NO: 31), wherein
Y is tyrosine,
R1 is an amino acid selected from the group of tryptophan (W) or alanine (A),
R2 is an amino acid selected from the group consisting of leucine (L) or threonine (T),
R3 is an amino acid selected from the group consisting of leucine (L), alanine (A), aspartic acid (D), phenylalanine (F), or tyrosine (Y),
R8 is leucine (L),
R4 is an amino acid selected from the group consisting of leucine (L), glycine (G) or threonine (T),
R5 is an amino acid selected from the group consisting of phenylalanine (F), leucine (L) or methionine (M), and
R9 is valine (V) or leucine (L); or
c) K-D-R6-M-V-R7-F-K-D (SEQ ID NO: 2), wherein
K is lysine,
D is aspartic acid,
R6 is an amino acid selected from the group consisting of leucine or aspartic acid,
M is methionine,
R7 is selected from the group consisting of Glutamine-Tryptophan (Q-W) and arginine-phenylalanine (R-F), and
F is phenylalanine;
or a pharmaceutically acceptable salt thereof.

2. The CPP-IRF5 peptide according to claim 1, wherein the peptide comprises an amino acid sequence of 20 to 40 amino acids and wherein said amino acid sequence further comprises, in part, an amino acid sequence motif selected from the group consisting of:
a) Y-R1-R2-R3-L-R4-R5-V (SEQ ID NO: 1), wherein
Y is tyrosine,
R1 is an amino acid selected from the group of tryptophan (W) or alanine (A),
R2 is an amino acid selected from the group consisting of leucine (L) or threonine (T),
R3 is an amino acid selected from the group consisting of leucine (L), alanine (A), aspartic acid (D), or phenylalanine (F),
L is leucine,
R4 is an amino acid selected from the group consisting of leucine (L), glycine (G) or threonine (T),
R5 is an amino acid selected from the group consisting of phenylalanine (F), leucine (L) or methionine (M), and
V is valine; or
b) K-D-R6-M-V-R7-F-K-D (SEQ ID NO: 2), wherein
K is lysine,
D is aspartic acid,
R6 is an amino acid selected from the group consisting of leucine or aspartic acid,
M is methionine,
R7 is selected from the group consisting of Glutamine-Tryptophan (Q-W) and arginine-phenylalanine (R-F), and
F is phenylalanine;
or a pharmaceutically acceptable salt thereof.

3. The CPP-IRF5 peptide according to claim 1, wherein the peptide comprises an amino acid sequence of 20 to 35 amino acids.

4. The CPP-IRF5 peptide according to claim 1, wherein the amino acid sequence motif is I-x-L-x-I-S-x-P-x-x-K (SEQ 1D NO: 30), wherein x is as defined in claim 1.

5. The CPP-IRF5 peptide according to any of claim 1, wherein the amino acid sequence motif is Y-R1-R2-R3-R8-R4-R5-R9 (SEQ ID NO: 31), wherein R1, R2, R3, R4, R5, R8 and R9 are as defined in claim 1.

6. The CPP-IRF5 peptide according to claim 1, wherein the amino acid sequence motif is Y-R1-R2-R3-L-R4-R5-V (SEQ ID NO: 1), wherein R1, R2, R3, R4 and R5 are as defined in claim 2.

7. The CPP-IRF5 peptide according to claim 1, wherein the amino acid sequence motif is MANLG-Y-R1-R2-R3-L-R4-R5-V (SEQ ID NO: 3), wherein M is methionine, A is alanine, N is asparagine, L is leucine, G is glycine, Y is tyrosine, V is valine and R1, R2, R3, R4, and R5 are as defined in claim 1.

8. The CPP-IRF5 peptide according to claim 1, wherein the amino acid sequence motif is MANLG-Y-R1-R2-R3-L-R4-R5-V (SEQ ID NO: 3), wherein M is methionine, A is alanine, N is asparagine, L is leucine, G is glycine, Y is tyrosine, V is valine and R1, R2, R3, R4, and R5 are as defined in claim 2.

9. The CPP-IRF5 peptide according to claim 1, wherein the amino acid sequence motif is K-D-R6-M-V-R7-F-K-D (SEQ ID NO: 2), wherein R6 and R7 are as defined in claim 2.

10. The CPP-IRF5 peptide according to claim 1, additionally comprising a second peptide which is a cell penetrating peptide (CPP).

11. The CPP-IRF5 peptide according to claim 1, additionally comprising an N-terminal modification, a C-terminal modification or both.

12. The CPP-IRF5 peptide according to claim 1, additionally comprising an N-terminal modification selected from acetylation, a C-terminal modification selected from amidation or both.

13. The CPP-IRF5 peptides according to claim 1, wherein the peptides comprise an amino acid sequence selected from the group consisting of:
SEQ ID NO 13:
IRLQISNPYLKFIPLKRAIWLIK,
SEQ ID NO 14:
MIILIISFPKHKDWKVILVK,
SEQ ID NO 4:
MANLGYWLLLLFVTMWTDVGLAKKRPKP,
SEQ ID NO 5:
MANLGYWLALLFVTMWTDVGLFKKRPKP,
SEQ ID NO 8:
KDLMVQWFKDGGPSSGAPPPS,
SEQ ID NO 9:
IRLQISNPDLKDLMVQWFKDGGPSSGAPPPS,
and
SEQ ID NO 10:
PFPPLPIGEEAPKDDMVRFFKDLHQYLNVV.

14. A cell-penetrating peptide which binds interferon regulatory factor IRF5 (CPP-IRF5), wherein the peptide comprises an amino acid sequence of 20 to 40 amino acids selected from the group consisting of:
SEQ ID NO 6:
MANLGYWLLALFVTYWTDLGLVKKRPKP,
SEQ ID NO 7:
MANLGYWLYALFLTMVTDVGLFKKRPKP,
or a pharmaceutically acceptable salt thereof.

15. The CPP-IRF5 peptides according to claim 1, wherein the peptides comprise amino acid sequence SEQ ID NO 13: IRLQISNPYLKFIPLKRAIWLIK.

16. A method for screening peptides that inhibit IRF5, comprising the steps of:
a) providing a peptide to be tested;
b) diluting said peptide in solution;
c) preparing a first buffered solution comprising biotin-IRF5 and His-IRF5, wherein each IRF-5 is a mixture of monomer and dimer;
d) combining the diluted peptide solution of step b) with the buffered solution of step c) and incubating at room temperature;
e) preparing a second buffered solution comprising Eu conjugated streptavidin, as a fluorescence donor, and APC (allophycocyanin) labeled anti-His Ab, as a fluorescence acceptor, for detecting biotin-IRF5 and His-IRF5 dimer formation;
f) combining the second buffered solution of step e) with the combined solutions of step d) and incubating at about 4 degrees C. for about 1 day; and
g) determining dimer formation via FRET assay, wherein a decreased FRET signal, as compared to a control group, shows inhibition of IRF5 dimer formation by the peptide.

17. The method of claim 16, wherein the FRET assay is a homogeneous time-resolved fluorescence resonance energy transfer (TR-FRET) assay.

18. The method of claim 16, wherein the IRF5 is selected from the group consisting of mutant S430D (222-467) and Wild type IRF5 (222-467).

19. A pharmaceutical composition, comprising one or more CPP-IRF5 peptides according to claim 1 or a pharmaceutically acceptable salt thereof and one or more pharmaceutically acceptable excipients.

20-21. (canceled)

22. A method for the treatment or prevention of systemic lupus erythematosus (SLE) or other autoimmune diseases wherein IRF5 signaling plays a significant role, comprising the step of administering a CPP-IRF5 peptide according claim 1 or a pharmaceutically acceptable salt thereof to a subject in need thereof.

23-24. (canceled)

25. A cell-penetrating peptide which binds human interferon regulatory factor IRF5 (CPP-IRF5), wherein the peptide comprises an amino acid sequence selected from the group consisting of SEQ ID NOS: 4, 5, 6, 7, 8, 9, 10, 13 and 14.

26. A cell-penetrating peptide which binds interferon regulatory factor IRF5 (CPP-IRF5), wherein the peptide comprises an amino acid sequence of at least 20 to about 35 amino acids, wherein said amino acid sequence further comprises, in part, an amino acid sequence motif selected from the group consisting of:
a) Y-R1-R2-R3-L-R4-R5-V (SEQ ID NO: 1),
wherein Y is tyrosine (Tyr), R1 is an amino acid selected from the group of tryptophan (Trp) and alanine (Ala), R2 is an amino acid selected from the group consisting of leucine (Leu) and threonine (Thr), R3 is an amino acid selected from the group consisting of leucine (Leu), alanine (Ala), aspartic acid (Asp) and phenylalanine (Phe), L is leucine (Leu), R4 is an amino acid selected from the group consisting of leucine (Leu), glycine (G) and threonine (Thr), R5 is an amino acid selected from the group consisting of phenylalanine (Phe), leucine (Leu) and methionine (Met), and V is valine (Val); and
b) K-D-R6-M-V-R7-F-K-D (SEQ ID NO: 2),
wherein K is lysine (Lys); D is aspartic acid (Asp), R6 is an amino acid selected from the group consisting of leucine (Leu) and aspartic acid (Asp), M is methionine (Met), R7 is selected from the group consisting of Q-W and R-F, and F is phenylalanine (Phe).

27. The cell-penetrating peptide of claim 26, wherein the amino acid sequence motif is MANLG-Y-R1-R2-R3-L-R4-R5-V (SEQ ID NO: 3).

28. The cell-penetrating peptide of claim 27, wherein the peptide comprises an amino acid sequence selected from the group consisting of SEQ ID NOS 4, 5, 6 and 7.

29. The cell-penetrating peptide of claim 26, wherein the peptide comprises an amino acid sequence selected from the group consisting of SEQ ID NOS 8, 9 and 10 and wherein further the amino acid sequence motif is K-D-R6-M-V-R7-F-K-D (SEQ ID NO: 2).

30. An isolated and purified peptide of at least 20 to about 40 amino acids, consisting of a first and an optional second polypeptide, wherein the first peptide:
i. comprises an amino acid sequence of at least 20 amino acids,
ii. has the ability to bind interferon regulatory factor 5 (IRF5),
iii. and wherein the first peptide comprises, in part, an amino acid motif of K-D-R6-M-V-R7-F-K-D (Seq ID NO. 2)
and the optional second peptide is a cell penetrating peptide (CPP).

31. An isolated and purified peptide of at least 20 to about 40 amino acids, consisting of a first and an optional second polypeptide, wherein the first peptide:
i. comprises an amino acid sequence of at least 20 amino acids,
ii. has the ability to bind human interferon regulatory factor 5 (IRF5),
iii. and wherein the first peptide comprises an amino acid sequence of SEQ ID NOS:8-10
and the optional second peptide is a cell penetrating peptide (CPP).

32. (canceled)
